(19) United States
(12) Patent Application Publication
(10) Pub. No.: US 2011/0206739 A1
(43) Pub. Date: Aug. 25, 2011

(54) COMPOSITIONS AND METHODS FOR THE PREPARATION OF NANOEMULSIONS

(75) Inventors: Robert J. Nicolosi, Nashua, NH (US); Fonghsu Kuo, Nashua, NH (US); SriKanth Kakumanu, Lowell, MA (US); Shawn Konecni, Nashua, NH (US); Carl W. Lawton, Chelmsford, MA (US)

(73) Assignee: UNIVERSITY OF MASSACHUSETTS, Boston, MA (US)

(21) Appl. No.: 12/935,180

(22) PCT Filed: Mar. 30, 2009
(86) PCT No.: PCT/US2009/038835

§ 371 Date: Apr. 6, 2011

Related U.S. Application Data
(60) Provisional application No. 61/040,482, filed on Mar. 28, 2008.
(60) Provisional application No. 61/144,237, filed on Jan. 13, 2009.
(60) Provisional application No. 61/144,246, filed on Jan. 13, 2009.

(51) Int. Cl. A61K 9/107; A61K 31/37; A61K 31/12; A61K 31/513; A61K 31/655; A61K 31/337; A61K 31/352; A61K 31/045; A61K 38/28; A61P 5/50; A61P 39/06; A61P 35/00

(52) U.S. Cl. 424/400; 514/457; 514/679; 514/274; 514/151; 514/449; 514/456; 514/729; 514/5.9

(57) ABSTRACT

The disclosure relates to compositions and methods of forming nanoemulsions, e.g., containing an active component, in combination with lipophilic components such as oils, hydrophilic components such as water, and one or more surfactants capable of causing a temperature-dependent phase inversion, such as a nonionic polyethoxylated surfactant. Nanoemulsions containing the active component can be produced having average oil droplet sizes of less than 100 nm, 50 nm, or 25 nm without the need for high energy emulsion forming methods (such as microfluidization) by combining the surfactant and the oil in specified weight ratios (e.g., at least 3:1) prior to forming the nanoemulsion.

### RELATED APPLICATIONS

This application claims priority from each of Provisional U.S. Patent Application Nos. 61/040,482 (filed on Mar. 28, 2008), 61/144,237 (filed Jan. 13, 2009) and 61/144,246 (filed Jan. 13, 2009), and the subject matter of each is incorporated herein by reference in its entirety.

### TECHNICAL FIELD

This disclosure relates to nanoemulsions, including compositions and processes for the manufacturing nanoemulsions and compositions containing nanoemulsions.

### BACKGROUND

Nanoemulsions have been studied for numerous applications, including delivery of various additional components such as pharmaceutical, nutraceutical or cosmeceutical agents. For example, nanoemulsions offer a potential substitute for the formulation of poorly soluble drugs. Nanoemulsions are typically transparent or translucent kinetically stable compositions of suspended oil or water droplets or particles having diameters that can be less than about 100-300 nm (in contrast to microemulsions having a thermodynamic equilibrium between components present in different phases). Compared to typical micron-sized emulsion preparations, which can have particles that are thousands of nanometers in size, nanoemulsion systems with smaller particle sizes and increased stability can provide increased bioavailability and efficacy for delivering a number of bioactive compounds such as anti-inflammatory agents, insulin and other drugs.

Nanoemulsions can be formed by two different types of processes: high energy emulsification methods and low energy phase inversion temperature methods. In high energy emulsion forming methods, a mixture of nanoemulsion components (e.g., oil, water, surfactant and an optional pharmaceutical, nutraceutical or cosmeceutical agent) is subjected to a continuous turbulent flow at high pressure (e.g., at least 25,000 psi) to form the nanoemulsion using a microfluidizer apparatus (e.g., as described by Cook et al. in U.S. Pat. Nos. 4,533,254 filed on Feb. 21, 1984, and 4,908,154, filed on May 26, 1987). However, nanoemulsions formed by high energy emulsion forming methods can have varying degrees of stability and relatively non-uniform or larger particle sizes (e.g., particle size distributions having multiple peaks).

Alternatively, nanoemulsions can be formed by low energy “self-assembly” methods without microfluidizer processing by combining a surfactant capable of temperature-dependent phase inversion (e.g., a nonionic polyethoxylated surfactant) with other nanoemulsion components (e.g., oil, water and an optional pharmaceutical, nutraceutical or cosmeceutical agent). The nanoemulsion components are mixed and heated above a phase inversion temperature (PIT) of the surfactant (i.e., the temperature at which the affinity of the surfactant for the different phases changes). For example, an oil-in-water (O/W) macro-emulsion can undergo a reversible, temperature-dependent transitional phase inversion above the PIT to form a water-in-oil (W/O) emulsion. Subsequent rapid cooling of the W/O emulsion below the PIT, for example by thermal cooling or adding water, can result in the formation of a kinetically stable O/W nanoemulsion composition of oil droplets (optionally containing the pharmaceutical, nutraceutical or cosmeceutical agent) suspended in water. Thermal cooling to form a nanoemulsion can be performed, for example, by placing the vessel containing the W/0 emulsion in an ice bath. Unless otherwise indicated, “rapid cooling” refers to cooling at a rate suitable to form a nanoemulsion without microfluidization.

However, there remains a need for nanoemulsion compositions and methods for producing nanoemulsions having reduced oil droplet size and/or improved droplet size uniformity. Also needed are compositions and methods for producing nanoemulsions that allow a predictable reduction in droplet size as a function of composition. In addition, there is a need for forming nanoemulsions from compositions with less than 20 wt % oil and levels of surfactant that are suitable for an intended use and are acceptable to applicable regulatory agency requirements, such as formulation of consumer products.

In addition, there remains a need for methods of forming nanoemulsion formulations containing active substances and having small droplet sizes for delivery of the substances. There is also a need for methods of formulating such nanoemulsion compositions in a manner that is stable and includes an amount of surfactant that is acceptable for an intended use. Such nanoemulsions can provide kinetically stable delivery vehicles with enhanced bioavailability for delivery of an active substance in the nanoemulsion.

### SUMMARY

This disclosure describes compositions and methods useful for forming nanoemulsions having desirable droplet sizes of a lipophilic substance suspended in a hydrophilic substance (e.g., an aqueous carrier containing average droplets of oil) with average sizes less than about 100, 50, or 25 nm. The compositions can include oil, water, and a surfactant capable of temperature-dependent phase inversion between the oil and water phases, such as a nonionic polyethoxylated surfactant. Such compositions can be used to form nanoemulsions by temperature-dependent phase inversion of the surfactant. As described herein, nanoemulsions formed by temperature-dependent phase inversion of a surfactant are also termed self-assembled nanoemulsions (“SANE”).

Certain embodiments of the invention are based on the discovery that the average droplet size in a nanoemulsion can be controlled (e.g., reduced to below 100 nm) by varying a weight ratio between two components (e.g., the oil and surfactant) in the composition used to form the nanoemulsion when the amount of lipophilic component in the composition is maintained below about 5 wt %. For example, compositions having about 2 wt % of an oil with surfactant to oil weight ratios of 5:1 or 7:1 can be used to form emulsions having an average droplet or particle size of about 25 nm or less, without microfluidization. In addition, certain nanoemulsions can be formed from a composition containing an active component (e.g., a pharmaceutical, nutraceutical, and/or a cosmaceutical).

The SANE compositions with an average droplet or particle size of less than 100 nm (including average particles sizes of less than 50 nm, and/or less than 25 nm) can be formulated with less than 5, 4, or 3 wt % oil, including compositions with about 2 wt % oil. The droplet or particle size of a lipophilic component in the nanoemulsion can be decreased by decreasing the Ros ratio in the first composition (defined as the weight ratio of the total lipophilic component to the total weight of the lipophilic component and the surfactant) to about 0.500 and below (e.g., Ros ratios of 0.125-0.500). The SANE compositions can be characterized by one or more of the following weight ratios: an oil/(oil+surfactant) ratio of about 0.500 or less (including 0.250, 0.167 or 0.125); water/(water+oil) weight ratios of about 0.980 or less (including ratios of 0.979, 0.978 and 0.977); oil/(oil+water) weight ratios of about 0.023 or less (including 0.022, 0.021, and 0.020); and/or water/(water+surfactant) weight ratios of 0.143 or less (including 0.102, 0.061 or 0.020). In addition, the weight ratio between the surfactant and the lipophilic component, and/or the combination of active component, the surfactant and the lipophilic component can be selected to form a nanoemulsion having an average lipophilic component droplet size of up to 100 nm. This can be done without microfluidization or other high energy emulsion forming procedures.

In addition, some embodiments of the invention are based on the discovery that SANE compositions do not form when certain active components are added to compositions of certain lipophilic components, hydrophilic components and surfactants that otherwise form a stable SANE composition in the absence of the additional active component. In particular, some embodiments are based, in part, on the discovery that certain active components (e.g., dacarbazine, gemcitabine or coumarin), do not form stable SANE compositions from a rice bran oil lipophilic component in combination with a polyethylene glycol 660 hydroxystearate surfactant (e.g., Solutol® HS15), while other active components (e.g., paclitaxel, tocotrienols, or coumarin) are able to form SANE compositions with these components. Furthermore, other embodiments of the invention are based in part on the discovery that certain active components (e.g., lutein, tocotrienols), do not form stable SANE compositions from a rice bran oil lipophilic component in combination with a C20 ethoxylated monoglyceride surfactant (e.g., EMG-20).

In one embodiment, the disclosure describes methods of forming a self-assembled nanoemulsion including the steps of (a) combining a lipophilic component (e.g., a non-toxic oil), an active component, a hydrophilic component (e.g., water) and a surfactant having a phase inversion temperature (PIT) between the lipophilic and hydrophilic components in a first composition containing less than about 5% by weight of the lipophilic component and having a weight ratio (Ros) of the lipophilic component to the total weight of the surfactant in the first composition is about 0.5 or less, and (b) heating the first composition above the PIT of the surfactant for a time sufficient to cause at least a portion of the mixture to undergo a phase inversion to form a second composition; and (c) cooling the second composition to form a stable nanoemulsion having droplets of the lipophilic component of an average droplet or particle size of up to 100 nm suspended in the hydrophilic component. The surfactant is selected to cause a temperature dependent phase inversion between the lipophilic component and the hydrophilic component at or above a surfactant PIT. The lipophilic component can include, but is not be limited to, a non-toxic oil (e.g., vegetable oil, coconut oil, soybean oil, flax seed oil, rice bran oil, fish oil, and the like). The active component(s) can be dissolved in the lipophilic component. Nanoemulsions with desirably small droplet particle sizes (e.g., less than 50 nm, 25 nm or smaller) can be obtained using formulations with surfactant to lipophilic component weight ratios of 3:1, 4:1, 5:1, 6:1, 7:1 or greater. In addition, the weight ratio between the surfactant and the lipophilic component, and/or the combination of active component, the surfactant and the lipophilic component can be selected to form a nanoemulsion having an average lipophilic component droplet size of up to 100 nm. This can be done without microfluidization or other high energy emulsion forming procedures.

In another embodiment, the disclosure provides methods of forming self-assembled nanoemulsions including one or more active components (e.g., pharmaceuticals, nutraceuticals, and the like), as well as methods of treatment including the administration of these SANE compositions. The active components can be bioactive materials incorporated into the nanoemulsions by combining (e.g., dissolving) the active component in the lipophilic component prior to or during one or more steps in the nanoemulsion formation process described herein. The active component can be dissolved in the lipophilic component prior to combination with the hydrophilic component. The resulting nanoemulsion can include the hydrophilic component within droplets or particles of the lipophilic component in the nanoemulsion. The SANE compositions can be nanoemulsions or stable water dispersions of water soluble compounds.

Methods of treatment can include administration of the nanoemulsions including the active component in a therapeutically appropriate manner, which can include administration in a manner commensurate with administration of the active component separate from the nanoemulsion. The active component can be therapeutically effective at lower dosages when formulated in a nanoemulsion than when delivered in a carrier that is not a nanoemulsion.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Unless otherwise indicated, the terms “microfluidized,” “microfluidizing,” “microfluidization,” or “microfluidizer” as used herein refer to an instrument or a process that utilizes a turbulent flow at high pressure including, but not limited to, a microfluidizer or other like device that may be useful in creating a uniform nanoemulsion. For example, microfluidizing can create a uniform nanoemulsion comprising a pharmaceutical, nutraceutical or cosmeceutical from a premix within a thirty second time frame (typically referred to a single pass exposure). Typically, a microfluidizer can be operated at a pressure of approximately 25,000 PSI to generate a uniform nanoemulsion.

Unless otherwise indicated, the term “active component” as used herein refers to any nutraceutical, pharmaceutical, nutraceutical or cosmeceutical that can be in a nanoemulsion using the methods for preparing nano-emulsions described herein. Examples of nutraceuticals include, but are not limited to, polyphenols (e.g., curcumin), flavenoids (e.g., quercetin), carotenoids (e.g., lutein), tocopherols and/or tocotrienols (e.g., Vitamin E). Pharmaceuticals that can be used as the active component can include, but are not limited to, selective estrogen receptor modulators (SERM) (e.g., tamoxifen), alkylating agents (e.g., substituted imidazole compounds such as dacarbazine), taxane compounds (e.g., paclitaxel), a nucleoside analog (e.g., gemcitabine), a statin (e.g., lovastatin, atorvastatin, simvastatin, and the like), a pyrimidine analog (e.g., 5-fluorouracil), and the like. Cosmeceuticals include for example, injectable bulking agents (e.g., collagen-based injectable materials for cosmetic applications), pentapeptides, anti-wrinkling formulations such as cis-retinoic acid, and hydroxy acids.

Unless otherwise indicated, “average particle size” refers to the z-average particle or droplet diameter measured by dynamic laser light scattering, also called Photon correlation spectroscopy (e.g., using the Malvern Zetasizer-S instrument, Malvern Instruments Inc., Southborough Mass.). Unless otherwise indicated, the z-average particle sizes were determined using the Malvern Zetasizer-S instrument with a 4 mW He—Ne laser operating at a wavelength of 633 nm and an avalanche photodiode detector (APD). The z-average diameter is the mean hydrodynamic diameter and the polydispersity index is an estimate of the width of the distribution. Both z-average diameter and polydispersity index (“PDI”) are calculated according to the International Standard on dynamic light scattering, ISO13321.

Other features, objects, and advantages of the invention will be apparent from the description, drawings, and from the claims.

### DESCRIPTION OF DRAWINGS

FIG. 1 is a diagram illustrating a method of preparing a self-assembled nanoemulsion.

FIG. 2 is a graph plotting the average particle size of nanoemulsions formulated using rice bran oil as the lipophilic component, combined with different amounts of different surfactants and water as the hydrophilic component. The S/O ratio refers to the initial weight ratio of surfactant to rice bran oil mixed before adding water.

FIG. 3 is a graph plotting the average particle size of nanoemulsions formulated using coconut oil as the lipophilic component, combined with different amounts of different surfactants and water as the hydrophilic component. The S/O ratio refers to the initial weight ratio of surfactant to coconut oil mixed before adding water.

FIG. 4 is a graph plotting the average particle size of nanoemulsions formulated using soybean oil as the lipophilic component, combined with different amounts of different surfactants and water as the hydrophilic component. The S/O ratio refers to the initial weight ratio of surfactant to soybean oil mixed before adding water.

FIG. 5 is a graph plotting the average particle size of nanoemulsions formulated using fish oil (e.g., omega three cod liver oil) as the lipophilic component, combined with different amounts of different surfactants and water as the hydrophilic component. The S/O ratio refers to the initial weight ratio of surfactant to fish oil mixed before adding water.

FIG. 6 is a graph showing the particle size distribution for three self-assembled nanoemulsions (i.e., SANE compositions) formed from water, a Solutol® HS15 surfactant and fish oil with a 5:1 S/O ratio.

FIG. 7 is a graph showing the particle size distribution for three SANE compositions formed from water, a Solutol® HS15 surfactant and vegetable oil with a 2:1 S/O ratio.

FIG. 8A is a graph showing the effect of different oils formulated via PIT nanoemulsion on colon cancer (CCL-221) cell uptake.

FIG. 8B is a graph showing the effect of different oils formulated via PIT nanoemulsion on melanoma cancer (Melma-3m) cell uptake.

FIG. 8C is a graph showing the effect of different oils formulated via PIT nanoemulsion on cervical cancer (CCL-2) cell uptake.

FIGS. 9A and 9B are a pair of transmission electron micrographs: (A) a TEM image of the DMSO prep of Curcumin (note clumping and irregular disorganized structures) and (B) the nanoemulsion (SANE) preparation of curcumin (note small particle size of about 20 nm and homogeneity of population).

FIG. 9C is a particle size distribution measurement showing the oil/curcumin droplet size for a SANE composition.

FIG. 10A is a plot of the electrokinetic potential of the curcumin SANE composition.

FIG. 10B is a plot of the zeta potential (electrokinetic potential) of curcumin in water.

FIG. 11 is a graph showing the in vitro activity of both the curcumin nanoemulsion and the curcumin-DMSO mixture against melanoma cancer cells.

FIG. 12A is a graph of the particle size distribution of a 2.5 mM 5-FU SANE composition.

FIG. 12B is a graph of the particle size distribution of a 10 nM 5-FU SANE composition.

FIG. 13A is a graph showing the effect of 5-FU nanoemulsion on cell proliferation of colon cancer cell lines (CCL-221). Compared to non-nanoemulsion of 5-FU, 5-FU nanoemulsion prevented cell proliferation of CCL-221 at 24 μM (−35%, p<0.001) and 12 μM (−25%, p=0.011). For all groups, N=9 and error bar was represented by SEM.

FIG. 13B is a graph showing the effect of 5-FU nanoemulsion on cell proliferation of melanoma cancer cell lines (Melma-3m). Compared to non-nanoemulsion 5-FU, 5-FU nanoemulsion prevented cell proliferation of Melma-3m up to 24% at 24 μM (p<0.001). For all groups, N=9 and error bar was represented by SEM.

FIG. 13C is a graph showing the effect of 5-FU nanoemulsion on cell proliferation of cervical cancer cell lines CCL-2). For all groups, N=9 and error bar was represented by SEM.

FIG. 14 is a graph of a particle size distribution measured for a dacarbazine SANE composition.

FIG. 15A is a graph showing the effect of DTIC nanoemulsion on cell proliferation of colon cancer cell line CCL-221.

FIG. 15B is a graph showing the effect of DTIC nanoemulsion on cell proliferation of skin cancer cell line Malme-3M.

FIG. 16A is a graph of particle size distribution of SANE compositions including paclitaxel.

FIG. 16B is a graph of particle size distribution for a mixture of paclitaxel in DMSO.

FIG. 16C is a graph showing the percent inhibition of a paclitaxel SANE composition and a DMSO paclitaxel composition against PL-45 cells.

FIG. 16D is a graph showing the percent inhibition of a paclitaxel SANE composition and a blank SANE composition against CCL-221 cells.

FIG. 16E is a graph showing the percent inhibition of a paclitaxel SANE composition and a DMSO paclitaxel composition against CCL-221 cells.

FIG. 16F is a graph showing the percent inhibition of a paclitaxel SANE composition and a blank SANE composition against PL-45 cells.

FIG. 16G is a graph showing the percent inhibition of a paclitaxel SANE composition and a DMSO paclitaxel composition against P10.05 cells.

FIG. 16H is a graph showing the percent inhibition of a paclitaxel SANE composition and a blank SANE composition against P10.05 cells.

FIG. 17A is a graph of particle size distribution of SANE compositions including tocotrienols.

FIG. 17B is a graph of particle size distribution for a mixture of tocotrienols in DMSO.

FIG. 17C is a graph of particle size distribution for a mixture of tocotrienols in water.

FIG. 18 is a graph showing the inhibition of cholesterol observed when exposing the HepG cells to a SANE composition including tocotrienols, a mixture of tocotrienols and DMSO and no treatment of the HepG cells.

FIG. 19A is a graph of particle size distribution of SANE compositions including siRNA.

FIG. 19B is a graph of particle size distribution of SANE compositions including siRNA after freezing and thawing of the SANE composition.

FIG. 19C is a graph of particle size distribution of SANE compositions including siRNA after freezing and thawing of the SANE composition.

FIG. 20 is a fluorescence image of transfected HeLa cells after contact with the siRNA SANE composition.

FIG. 21 is graph showing hamster blood glucose levels after administration of a SANE composition including insulin as an active component.

FIG. 22 is a table summarizing in vitro effects of SANE compositions including tamoxifen, 5-FU and Curcumin on Malme Melanoma Cells, CCL-4 Colon Cancer Cells, HTB-20 Cells, MCF-7 Cells, PL-45 Pancreatic Cells and/or HeLa Uterine Cells.

FIG. 23 is a graph showing measured HMG CoA Reductase Enzyme Activity for a Lovastatin SANE composition.

FIG. 24 is a graph showing the particle size distribution of SANE compositions containing Coumarin 6.

FIG. 25 is a graph showing the relative fluorescence intensities of Coumarin 6 in different formulations.

### DETAILED DESCRIPTION

This disclosure describes methods of making nanoemulsions. The nanoemulsions are useful, for example, to deliver lipophilic substances, such as therapeutic or nutritional oils and/or cosmetic products. For example, nanoemulsions with small (e.g., less than 100 nm) lipophilic droplets or particles can provide improved homogeneity of a lipophilic substance, improved bioabsorption or digestion, and/or improved penetration number of the lipophilic substance into tissue including, but not limited to, skin or hair. Nanoemulsions can be formulated as nutritional supplements (e.g., an omega-three fish oil or vitamin supplement) or combined with carrier-bioactive compounds such as creams, liquids, gels and patches. Nanoemulsions can provide improved penetration through tissue of the lipophilic droplets or particles, improving the efficacy of a product, providing a controlled rate of delivery of the product into tissue, and/or prolonging the shelf life of a product by decreasing its degradation. Stable nanoemulsions are thus useful components in a variety of products.

The nanoemulsions can also be used as a means to deliver active compounds (including pharmaceuticals, nutraceuticals and cosmaceuticals) that are not readily water soluble with increased bioavailability.

### Formation of Nanoemulsions

Processes and compositions for the formation of nanoemulsions, particularly self-assembled nanoemulsions (SANE) are provided herein. A composition suitable for forming a SANE composition can include a lipophilic component, an active component, a hydrophilic component, and a surfactant characterized by a PIT with respect to the lipophilic and hydrophilic components. The active component is preferably more soluble in the lipophilic component than the hydrophilic component. These compositions can include up to about 5 wt % of the lipophilic component and have the surfactant and the lipophilic component present in an initial weight ratio of 3:1 or greater (e.g., 5:1-7:1 or greater) to provide nanoemulsions with reduced droplet sizes of the lipophilic component.

The active component(s) can be present in the droplets of the lipophilic component of the SANE composition. The active component(s) can be present in the droplets of the lipophilic component of the SANE composition. The droplet or particle size of a lipophilic component in the nanoemulsion can be decreased by decreasing the Ros ratio in the first composition (defined as the weight ratio of the total lipophilic component to the total weight of the lipophilic component and the surfactant) to about 0.500 and below (e.g., Ros ratios of 0.125-0.500). The SANE compositions can also be characterized by one or more of the following weight ratios: an oil/(oil+surfactant) ratio of about 0.500 or less (including 0.250, 0.167 or 0.125); water/(water+oil) weight ratios of about 0.980 or less (including ratios of 0.979, 0.978 and 0.977); oil/(oil+water) weight ratios of about 0.023 or less (including 0.022, 0.021, and 0.020); and/or water/(water+surfactant) weight ratios of 0.143 or less (including 0.102, 0.061 or 0.020). For example, SANE compositions having average droplet sizes of less than 100 nm, 50 nm or 25 nm can be obtained using compositions with a surfactant and lipophilic component (e.g., a non-toxic oil) in a weight ratio of 3:1 or 5:1 (“S/O ratio”).

In a composition having up to about 5 wt % of the lipophilic component containing the active component(s), increasing the weight ratio of the surfactant to the lipophilic component above about 3:1 can provide a reduction in the droplet size of the lipophilic component in the SANE composition where the Ros ratio in the first composition (defined as the weight ratio of the total lipophilic component to the total weight of the lipophilic component and the surfactant) is about 0.500 and below (e.g., Ros ratios of 0.125-0.500). Increasing the S/O ratio in an initial formulation from 1:1 to 3:1 can result in a reduction (e.g., about an 30-97% reduction) in the average droplet size (e.g., from about 1,000 nm down to about 30-619 nm) of the lipophilic oil component in the resulting SANE composition formed from the initial formulation; further increasing the S/O ratio in an initial formulation from 3:1 to 5:1 can result in about an additional reduction (e.g., a 70-83% reduction) of the average droplet size (e.g., down to about 21-121 nm) in a resulting SANE composition formed from the initial formulation according to the processes described herein. Further average droplet size reductions can be obtained in a SANE composition by increasing the S/O ratio from 5:1 to 7:1, which can result in an average droplet size of from about 15 to about 25 nm.

A nanoemulsion can be formed by heating a mixture of a lipophilic component and an active component to dissolve the active component in the lipophilic component. Next, the solution of the lipophilic component and the active component can be heated and mixed with a hydrophilic component and a surfactant characterized by a temperature dependent phase inversion between the lipophilic component and the hydrophilic component at or above a phase inversion temperature (“PIT”) of the surfactant, followed by rapidly cooling the mixture to below the PIT to form the nanoemulsion (e.g., cooling the mixture in a heat-conducting vessel placed in an ice bath until the contents of the vessel are at room temperature or about 25 degrees C.).

In one aspect, the nanoemulsion can consist essentially of, or consist of, the lipophilic component, the hydrophilic component, and the surfactant. For example, the nanoemulsion can be a three-component system with droplets or particles of the lipophilic component suspended in the hydrophilic component, with the surfactant associated with either or both components. The nanoemulsion can be formed in the absence of additional active components (such as additional pharmaceutical, nutraceutical, or cosmaceutical ingredients) that are not required to form the nanoemulsion. The nanoemulsion can be formed as a self-assembled nanoemulsion (SANE) formed without subjecting the nanoemulsion components to high energy emulsion forming methods (e.g., without microfluidization).

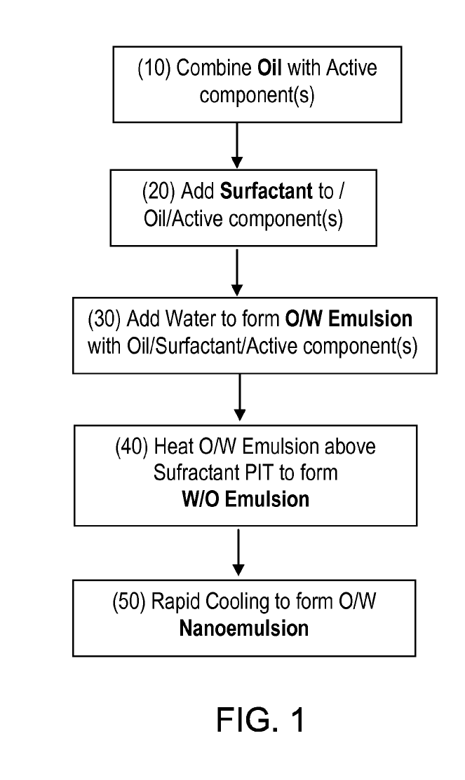

For example, a nanoemulsion can be formed by performing the steps shown in FIG. 1: (a) combining a lipophilic component (e.g., a non-toxic oil) and a surfactant (10), (b) combining the lipophilic component with a surfactant (20) that is characterized by a temperature dependent phase inversion between the lipophilic component and the hydrophilic component, (c) combining the mixture of the lipophilic component and the surfactant with a hydrophilic component (e.g., water) (30) to form an oil-in-water (“O/W”) emulsion; (d) heating the O/W emulsion above the phase inversion temperature of the surfactant (40) for a time sufficient to cause at least a portion of the mixture to undergo a phase inversion to reversibly form a water-in-oil (“W/O”) emulsion; and (e) cooling the second composition to form the nanoemulsion (50) having droplets of the lipophilic component of an average droplet size of up to 100 nm suspended in the hydrophilic component. An active component or ingredient (e.g., a pharmaceutical, nutraceutical and/or cosmaceutical) can be incorporated in the nanoemulsion, for example by dissolving the active ingredient in the lipophilic component prior to or during step (a) above to form a bioactive SANE composition.

Combining the mixture of the lipophilic component and surfactant with the hydrophilic component (30) can be performed by adding the hydrophilic component while heating the mixture below the PIT of the surfactant (e.g., to about 50-65° C.) while stirring the mixture (e.g., with a stir bar). Once formed, the O/W emulsion can be stirred and heated above the PIT (40) for a time period sufficient to mix the sample (e.g., about 5-15 minutes). Rapid cooling of the composition (e.g., a W/O emulsion) comprising the lipophilic component, the hydrophilic component and the surfactant is heated above the PIT of the surfactant can form the nanoemulsion.

Rapid cooling should include reducing the temperature of the composition at a rate sufficient to form the nanoemulsion, for example by placing a heat-conducting vessel containing the composition into an ice bath, or rapidly diluting the composition with a volume of the hydrophilic component (e.g., water) to reduce the temperature to below the PIT. Cooling can be performed until the temperature of the liquid in the reaction vessel is at about room temperature (e.g., about 25° C.), for example, by placing the heat-conducting vessel in ice water and adding room temperature water to the W/O emulsion. For example, the temperature of an O/W emulsion can be reduced at a sufficient rate in an ice bath, or by adding water at a temperature (e.g., about 20-30° C.) below the PIT in a volume equal to about 20-50% (including 20-30%) of the volume of the composition.

The droplet size of the lipophilic component in the nanoemulsion comprising the active component(s) can be reduced in a composition having a constant weight of up to about 5 wt % of the lipophilic component by combining the surfactant and the lipophilic component at increasing weight ratios above 1:1 (e.g., S/O weight ratios of about 3:1, 5:1 or 7:1, including non-integer fractional weight ratios there between). In some embodiments, the droplet size or particle size of a lipophilic component having up to about 5 wt % of the lipophilic component is decreased by increasing the weight ratio of the surfactant to the lipophilic component to about 3:1 or greater (e.g., about 3:1-10:1, 3:1-7:1, or 3:1-5:1) provides a reduction in the droplet size of the lipophilic component in the SANE composition. In other embodiments, the droplet size or particle size of a lipophilic component having up to about 5 wt % of the lipophilic component is decreased by decreasing the Ros ratio (defined as the weight ratio of the total lipophilic component to the total weight of the lipophilic component and the surfactant) to about 0.500 and below (e.g., Ros ratios of 0.125-0.500) in the composition of step (30) in FIG. 1.

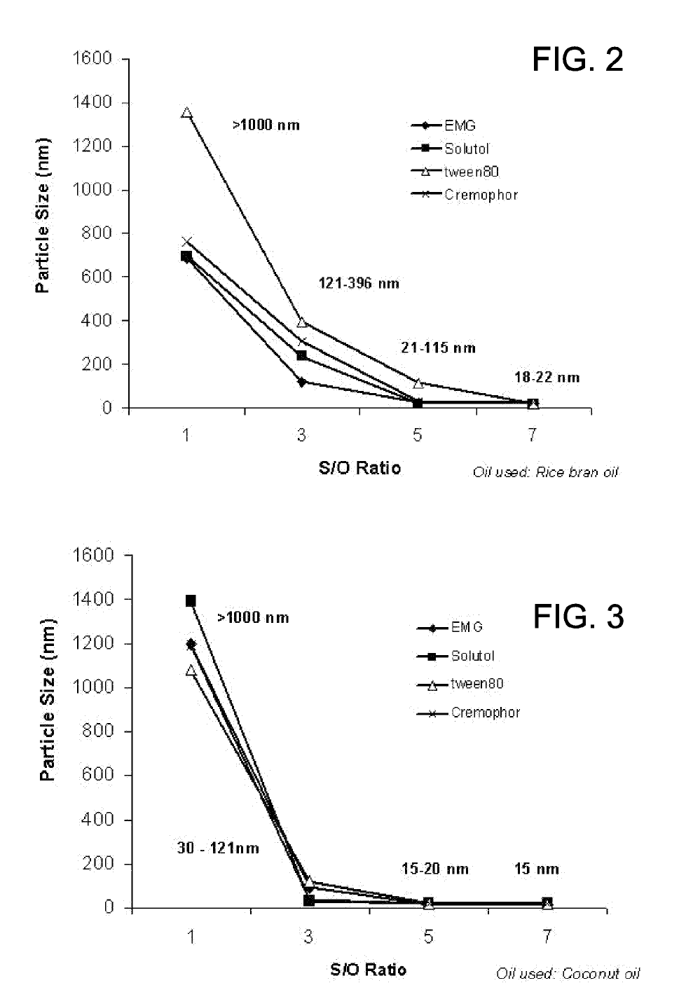

FIGS. 2-5 are graphs showing the average droplet size (also called particle size) as a function of the ratio of the surfactant to the lipophilic component (“S/O Ratio”) measured for nanoemulsions formed according to the method described with respect to FIG. 1 with 2 wt % oil, using different oil lipophilic components and different surfactants. The S/O ratio refers to the weight ratio of the surfactant to the oil initially combined in step (20) of FIG. 1. Each nanoemulsion is prepared without subjecting the O/W emulsion of step 30 of FIG. 1 to high energy emulsion forming methods (i.e., microfluidization was not used).

In some embodiments the volume of the entire system (oil+surfactant+water) is maintained at 50 ml. For example, with 1 g of oil, the droplet size of the oil in the nanoemulsion (step (50) in FIG. 1) can be decreased by increasing the initial weight ratio of the surfactant and the lipophilic component (“S/O ratio”) in the composition of step (20) of FIG. 1 above about 1:1, for example using S/O ratios of 2:1-10:1, 3:1-10:1, 5:1-10:1, 7:1-10:1, 3:1-7:1, 3:1-5:1, and 5:1-7:1, including 4:1, 5:1, 6:1 and any fractional ratios between 1:1 and 10:1. SANE compositions with up to 5 wt % of the lipophilic component and S/O ratios of 2:1, 3:1, 5:1, or 7:1 to 10:1 or greater, including ratios of 3:1, 5:1, 7:1 and ranges of S/O ratios between 1:1 and 10:1 can be formulated as, or added to, cosmetic and therapeutic compositions.

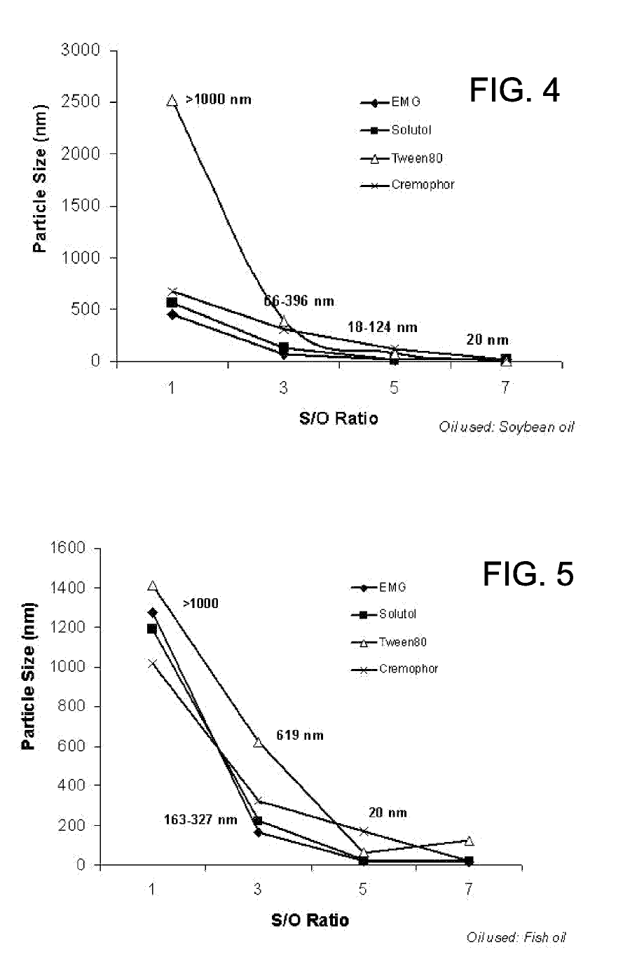

A total of sixty-four nanoemulsions were prepared, as described in Example 1, with 2 wt % oil and initial (i.e., before step (40) in FIG. 1) S/O weight ratios of 1:1, 3:1, 5:1 or 7:1. The average particle size of each nanoemulsion was plotted on one of the graphs shown in FIGS. 2-5. In each of these nanoemulsions, the average droplet size of the lipophilic component in the nanoemulsion decreased as the initial weight ratio of the surfactant to the oil was increased from 1:1 to 3:1 and from 3:1 to 5:1. In all but one of the sixty-four nanoemulsions (i.e., a SANE composition using a polyoxyethylenesorbitan monooleate surfactant sold under the tradename TWEEN® 80 used in obtaining some of the data in FIG. 5), further increasing the initial S/O weight ratio from 5:1 to 7:1 resulted in a nanoemulsion having lipophilic component droplet sizes that were the same or smaller than the corresponding nanoemulsions having the 5:1 S/O initial weight ratio.

Nanoemulsions having low average droplet sizes (e.g., below 25 nm) and polydispersity (e.g., droplet size measurement peak widths below about 10 nm) can be obtained using certain weight ratios of the surfactant and the lipophilic components described herein (including non-integer weight ratios between the exemplary weight ratios disclosed herein). The nanoemulsion droplets size distribution can be measured by light scattering using standard techniques and devices.

The surfactant is selected to cause a temperature dependent phase inversion between the lipophilic component and the hydrophilic component at or above a phase inversion temperature of the surfactant. A phase inversion occurs in a composition with fixed amounts of the surfactant, a lipophilic material, and a hydrophilic material, when the relative affinity of the surfactant between the lipophilic and hydrophilic materials changes by controlling the temperature. For example, an oil-in-water (O/W) macroemulsion composition of the lipophilic and hydrophilic materials combined with the surfactant can undergo a phase inversion to a water-in-oil (W/O) emulsion above a phase inversion temperature (PIT). The phase inversion can be reversible as a function of temperature near the PIT, unless the emulsion is rapidly cooled below the PIT to irreversibly form an O/W nanoemulsion.

The surfactant can be a nonionic polyethoxylated surfactant characterized by a temperature dependent phase inversion between a non-toxic oil lipophilic component and an aqueous hydrophilic component at or above the surfactant PIT. The PIT is a function of the chemical structure of the surfactant according to the Hydrophilic-Lipophilic Balance (HLB) number. The surfactant molecule can be nonionic. Nonionic surfactants find wide application in pharmaceutical, nutraceutical and cosmetic products and are usable over a wide range of pH values. In general, nonionic surfactant HLB values range from 1 to about 18 depending on their structure. Any nonionic surfactant causing a phase inversion at a PIT between the lipophilic and hydrophilic phases can be used. A lower HLB number corresponds to a more lipophilic surfactant; a higher HLB number corresponds to a more hydrophilic surfactant. Two or more surfactants can be used in compositions to form nanoemulsions. The HLB number of a mixture of two surfactants with x weight percent of a first surfactant (A) and y weight percent of a second surfactant (B) is given by the formula: HLB(A+B)=(Ax+Bx)/(x+y). The HLB temperature (T_{HLB}) of a system can be determined from the equation developed by Kunieda and Shinoda given below,

T_{HLB}=Kₒᵢₗ(N_{HLB}−Nₒᵢₗ)

where Kₒᵢₗ is approximately 17 degrees C./HLB unit for most alkanes and Nₒᵢₗ is a constant for given oil (oil number which decreases as the hydrocarbon molecular weight increases). See, e.g., Shinoda, K. and Saito, H., J. Colloid Interface Sci., 26, 70 (1968); Shinoda, K. and Saito, H., J. Colloid Interface Sci., 30, 258 (1969); Shinoda, K. and Kunieda, H., in Encyclopedia of emulsion Technology, Becer, P., Ed., Marcel Dekker, New York, Vol. 1, pp. 337-367 (1983), incorporated by reference herein as pertaining to methods of forming emulsions by phase inversion, and calculation of the HLB temperature for surfactants having a PIT.

Surfactants having a PIT suitable for use in forming a nanoemulsion include nonionic polyethoxylated surfactants. Some examples of suitable surfactants with a PIT when in contact with lipophilic and hydrophilic components include: ethoxylated mono- or diglycerides (e.g., a C20 ethoxylated monoglyceride such as the surfactant sold under the tradename EMG-20), polyoxyethylene esters of hydroxystric acids (e.g., a polyoxyethylene ester of 12-hydroxysteric acid such as the surfactant sold under the tradename SOLUTOL® HS15), a polyoxyethylene sorbitan monooleic acid ester (e.g., a Polyoxyethylene Sorbitan Monooleate such as the surfactant sold under the tradename TWEEN® 80), a polysorbate such as a Sorbitan monooleate (e.g., Span 80), or a polyoxyethylene oil (e.g., a polyoxyethylene castor oil such as the surfactant sold under the tradename CREMOPHOR EL®).

The lipophilic component can be a non-toxic liquid oil at room temperature (e.g., 25 degrees C.) and/or at the PIT of the surfactant. The oil can contain a mixture of mono- or polyunsaturated fatty acids and/or saturated fatty acids (e.g., linolenic acid, lauric acid, oleic acid, stearic acid, linoleic acid, myristic acid, caprylic acid, arachidic acid, behenic acid, palmitic acid and/or omega-3 fatty acids). For example, the lipophilic component can include vegetable oil, rice bran oil, fish oil (e.g., cod liver oil), coconut oil, and/or soybean oil. The lipophilic component can be a liquid at least at the surfactant PIT. The oils used in formulating the lipophilic component of the nanoemulsion can be selected to provide a composition suitable for a desired method of delivery. For example, medium chain fatty acids contained in coconut oil and palm kernel oil can passively diffuse from the GI tract to the portal system (longer fatty acids are absorbed into the lymphatic system) without requirement for modification like long chain fatty acids. Therefore, the emulsion delivery systems consisting of different oils potentially possess different delivery profile. Oils that can be used in the lipophilic component include, for example, soybean oil, corn oil, safflower oil, cottonseed oil, rice bran oil, flax oil, fish oil, and combinations thereof.

The hydrophilic component is a liquid at room temperature or at the PIT of the surfactant and can be selected to have a greater affinity for the surfactant than the lipophilic component above the PIT of the surfactant. The hydrophilic component can have a lower octanol-water partition coefficient than the lipophilic component, where the partition coefficient is the ratio of concentrations of un-ionized compounds between an octanol and a water phase. To measure the partition coefficient of ionizable solutes, the pH of the aqueous phase is adjusted such that the predominant form of the compound is un-ionized. The hydrophilic component can be or include, for example, water, an alcohol or a solution of water with one or more water-soluble materials. The hydrophilic and lipophilic components can be selected to have a desired affinity for the surfactant at a given temperature. The hydrophilic component can be an aqueous phase of the nanoemulsion, and can comprise demineralized or distilled water at an adequate percentage (q.s.p.) to achieve 100% of the formula, based on the total weight of the composition of the nanoemulsion.

### Incorporation of Active Components

Additional components can be optionally included in the nanoemulsion, such as one or more pharmaceutical, cosmeceutical, or nutraceutical materials, to form a bioactive SANE composition. Such components can be initially combined with the lipophilic component. For example, an active component, such as a pharmaceutical component, can be dissolved in an oil that is subsequently combined with a suitable surfactant and the hydrophilic component. Any pharmaceutical, cosmeceutical or nutraceutical having a solubility in the lipophilic component that permits subsequent formation of the nanoemulsion can be used.

An active component or ingredient (e.g., a pharmaceutical, nutraceutical and/or cosmaceutical) can be incorporated in the nanoemulsion, for example by dissolving the active ingredient in the lipophilic component prior to or during step (a) above to form a bioactive SANE composition. The bioactive SANE compositions including one or more active components can be made according to a method including any number of the following steps:

(a) Weigh a specified amount of a lipophilic component (e.g., 1 g, 2 wt %). The type of lipophilic component (e.g., oil) can depend on the active component (e.g., nutrient/drug) being formulated (e.g., the solubility of the active component and desired use of the composition). Specific examples of combinations of oils and active components are provided herein.

(b) Add the desired amount of one or more active components of interest to the lipophilic component to form a lipophilic mixture or to dissolve the active component in the lipophilic component.

(c) Heat and stir the active component into the lipophilic component for a suitable period of time (e.g., 5 minutes) in a desired manner (e.g., using a magnetic stirrer on a hot plate) until the active component visually appear to have dissolved in oil (e.g., heat to 50°-60° C.).

(d) Add a specified amount of a surfactant characterized by a PIT (e.g., a ethoxylated nonionic surfactant) in a desired weight ratio with the lipophilic component (e.g., 5 g, 10 wt %). The PIT (also referred to as HLB temperature) depends on the surfactant chemical structure, and can vary according to the HLB number (Hydrophile-Lipophile balance) of the surfactant. In general, PIT increases with increasing HLB number.

(e) Heat and stir the composition including the surfactant, lipophilic component, and the active component for a desired period (e.g., 5 minutes) at a temperature below the PIT (e.g., 50-60° C.) until the three components form a homogeneous mixture.

(f) Add distilled water to the composition including the surfactant, lipophilic component, and the active component and continue to mix and heat until an O/W macroemulsion forms. The water can be heated to the temperature of the mixture of the surfactant, lipophilic component, and the active component (e.g., 60° C.). For example, the O/W macroemulsion can have a total volume of the emulsion of about 50 ml.

(g) Heat the O/W macroemulsion from step (f) to and then above the PIT temperature of the surfactant. During heating, when the PIT (or HLB temperature) of the system is reached (65-70° C., phase inversion zone); the surfactant is in equilibrium with the oil and water phases. Heating the O/W macroemulsion above the PIT while stirring (e.g., up to 95° C.) inverts the system to a W/O emulsion. Once this stage is reached the heating and stirring is stopped.

(h) The W/O emulsion in step (g) is cooled rapidly (e.g., by placing vessel containing the W/O emulsion in ice water until the temperature is reduced to room temperature or about 25° C.) below the PIT, for example to room temperature (e.g., 25-30° C.) to obtain the O/W nanoemulsion (a kinetically stable SANE composition).

The nanoemulsions disclosed herein can be useful, for example, for enhancing the oral absorption of poorly soluble drug. Hydrophobic drugs can be dissolved in such systems, allowing them to be encapsulated as unit dosage forms for oral administration. Exemplary nanoemulsion formulations can be used to deliver an active agent to the gastrointestinal (GI) tract. A drug administered in this manner remains in solution in the GI tract, avoiding the dissolution step, which can limit the rate of absorption of hydrophobic drugs from the crystalline state. The nanoemulsions can be formulated with levels of surfactants that prevent GI side-effects as well as to a reduction in the free drug concentration and, thus, a reduced rate of intestinal absorption.

As described herein, the processes and compositions for producing nanoemulsions can provide nanoemulsions with reduced oil droplet size and increased droplet size uniformity (e.g., reduced average droplet size and reduced polydispersity index). The nanoemulsions can be characterized by a number of different parameters. For example, a nanoemulsion can be described in terms of a water/oil ratio defined as 100 times the weight ratio of (water)/(water+oil) in the initial W/O mixture (e.g., as described by Anton et al., “Design and production of nanoparticles formulated from nano-emulsion templates—A review,” Journal of Controlled Release, 128, 185-199 (2008)).

Preferably, the nanoemulsions contain more surfactant than oil by weight, with less than about 20 wt % oil. Izquierdo et al. have reported the formation of nanoemulsions by heating compositions containing a mixture of at least 20 wt % oil (e.g., decane, dodecane, tetradecane, hexadecane and isohexadecane) with water and a nonionic surfactant (e.g., 3.5 wt %) above the PIT temperature of the surfactant, where the weight ratio of the surfactant to oil of less than 1.0 (Izquierdo et al., “Formation and Stability of Nano-Emulsions Prepared Using the Phase Inversion Temperature Method,” Langmuir 18, 26-30 (2002); Izquierdo et al., “Phase Behavior and nano-emulsion Formation by the Phase Inversion Temperature Method,” Langmuir 20, 6594-6598 (2004)). In contrast, certain methods of making nanoemulsions can include combining a lipophilic component and a surfactant with a weight ratio of the surfactant to the lipophilic component being 1.0 or greater.

Certain methods of forming a nanoemulsion include preparing nanoemulsion compositions with less than 3.5 wt % (e.g., less than 3.0%) of a nonionic surfactant. Other examples of nanoemulsions are characterized by a weight ratio of oil/(water+oil) that is less than 0.2 (e.g., 0.02-0.20, including 0.02-0.03, 0.02-0.05, 0.02-0.10, and 0.02-0.15). The nanoemulsions can also have an oil/(oil+surfactant) weight ratio (“Ros value”) of less than 0.67, including ratios of 0.50 or less (e.g., 0.10-0.5, 0.2-0.5, 0.3-0.5 and 0.4-0.5). In contrast, Morales et al. report formation of nanoemulsions from compositions of mineral oil, water, and a nonionic surfactant (hexaethylene glycol monohexadecyl ether) with an oil/(water+oil) weight ratio of 0.2 by heating the mixture above the phase inversion temperature of the surfactant (Morales et al., “A Study of the Relation Between Bicontinuous Microemulsions and Oil/Water Nanoemulsion Formation,” Langmuir 19, 7196-7200 (2003)). However, the droplet size of these emulsions steadily increased as a when the ratio of oil/(oil+surfactant) was increased or decreased below 0.67 (Morales et al., id. at page 7199). For example, higher droplet sizes of about 75 nm were reported in compositions with a lower Ros value of 0.4 (id. at page 7199). These studies describe the formulation of nanoemulsions with at least about 20 wt % oil and oil-to-surfactant weight ratios that are less than 1.0.

In one example, a nanoemulsion includes coumarin (benzopyrone) or a derivative thereof, such as brodifacoum, bromadiolone, coumafuryl, and/or difenacoum, Ensaculin warfarin, and/or phenprocoumon (Marcoumar). For example, the nanoemulsion can include Coumarin 6 in a medical imaging dye formulation (e.g., a fluorescent dye or a contrast agent for magnetic resonance imaging). The nanoemulsion containing Coumarin 6 can be a fluorescent imaging dye (e.g., for imaging of macular degeneration) useful, for example, to detect abnormal cell proliferation (e.g., for detection of early and advanced metastatic cancers). Compared to dye formulations outside of the nanoemulsion, Coumarin 6 nanoemulsion dyes can have (a) improved stability and reduced levels of photolysis, (b) increased fluorescence intensity with lower noise (e.g., facilitating the detection of pathological processes using lower concentrations of Coumarin 6 to reduce side effects) and (c) increased water solubility. Example 2 illustrates the preparation of coumarin nanoemulsions. A nanoemulsion can include coumarin in a lipophilic component selected from the group consisting of soybean oil, coconut oil, cod liver oil or other fish oil, and/or rice bran oil. The coumarin nanoemulsion can include a polyoxyethylene ester of 12-hydroxysteric acid such as Solutol® HS15 in an S/O weight ratio with the lipophilic component of 1:1 to 5:1, and can be about 3:1 to 5:1 or about 5:1.

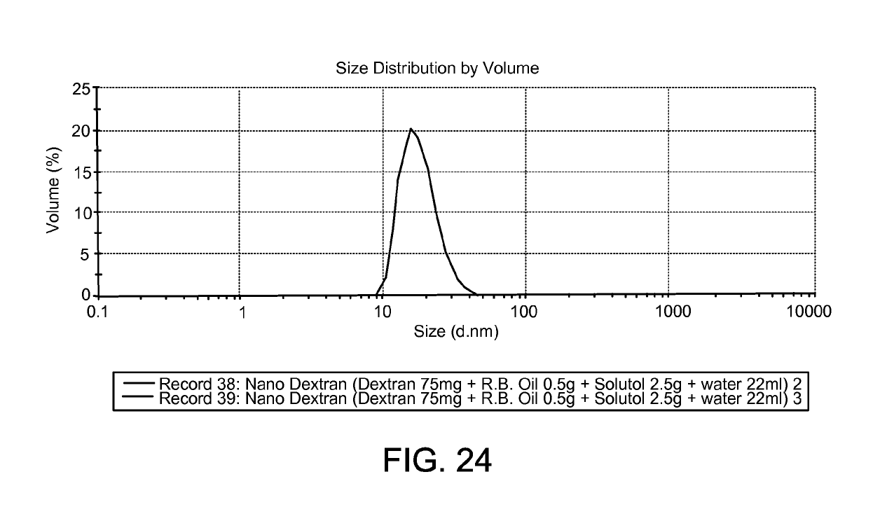

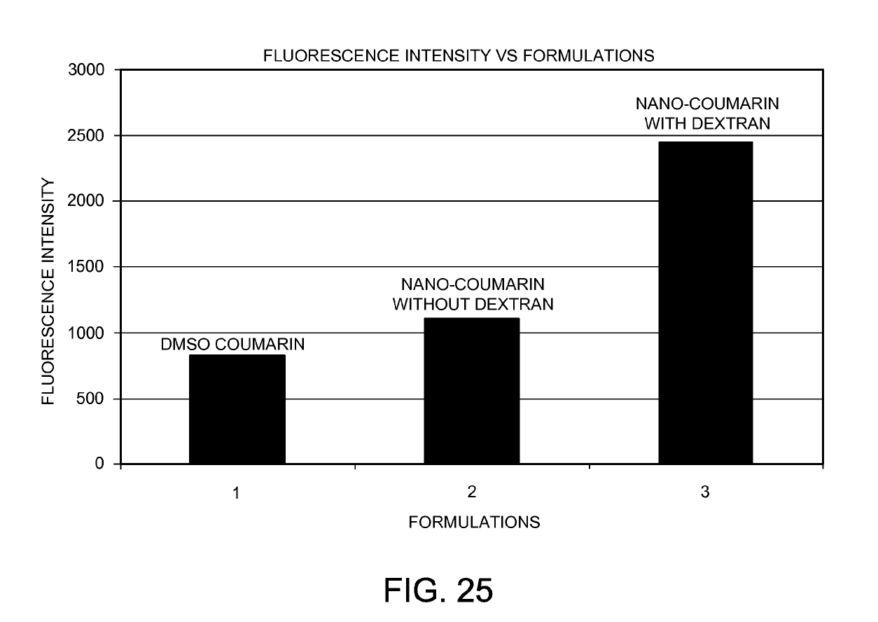

The fluorescence intensity of a Coumarin 6 nanoemulsion can be increased by adding a polysaccharide polymer such as dextran to the SANE formulation. As described in Example 2, the fluorescent dye Coumarin 6 SANE composition can optionally comprise dextran to increase fluorescence intensity of the SANE dye composition. Example 2 describes preparation of Coumarin 6 SANE dye formulations with and without the addition of the polymer dextran. Example 2 also describes measured particle size, stability and fluorescence intensity determined in samples of (a) DMSO coumarin 6 preparation, (b) nanoemulsion of coumarin 6 preparation without dextran, and (c) nanoemulsion of coumarin 6 with dextran. Data shown in FIGS. 24 and 25 and Table 2, show that a stable water dispersion of a nanoemulsion with or without dextran can be formulated with a particle size of about 25 nm or less.

As shown in FIG. 24, incorporation of dextran with a molecular weight of 1500 (75 mg dextran with 0.5 g rice bran oil, 2.5 grams Solutol® HS15 surfactant and 22 mL water) produced a nanoemulsion with an average particle size of 25 nm (Z-average particle size of 25.15 nm, peak width of 5.644 and PDI of 0.312). FIG. 25 shows the increase in fluorescence intensity of the fluorescent dye Coumarin 6. Table 2 describes the stability of the Coumarin preparations. As shown by the data in Table 2 is that over a 3 day period, these SANE preparations of Coumarin 6 were stable (determined by particle size changes) at room temperature for at least 3 days compared to the DMSO coumarin 6 preparation. Finally, the nanoemulsion of the coumarin 6 preparation containing dextran showed dramatic increases in fluorescence intensity compared to the DMSO and nanoemulsions without the dextran polymer.

In another example, a nanoemulsion includes a polyphenol, such as curcumin (i.e., (1E,6E)-1,7-bis(4-hydroxy-3-methoxyphenyl)-1,6-heptadiene-3,5-dione, CAS 458-37-7). Example 3 illustrates the preparation of a curcumin nanoemulsion and activity of the curcumin nanoemulsion against various cancer cell lines. A nanoemulsion can include a polyphenol such as curcumin in a lipophilic component selected from the group consisting of soybean oil, coconut oil, cod liver oil or other fish oil, and/or rice bran oil. The curcumin nanoemulsion can include a polyoxyethylene ester of 12-hydroxysteric acid such as Solutol® HS15 in a S/O weight ratio with the lipophilic component of 1:1 to 5:1, and can be about 3:1 to 5:1 or about 5:1.

In another example, a nanoemulsion includes a pyrimidine or pyrimidine analog such as 5-fluorouracil. Example 4 illustrates the preparation of such nanoemulsions using 5-fluorouracil (5-FU). A nanoemulsion can include the pyrimidine or pyrimidine analog in a lipophilic component selected from the group consisting of soybean oil, coconut oil, cod liver oil or other fish oil, and/or rice bran oil. The pyrimidine or pyrimidine analog nanoemulsion can include a polyoxyethylene ester of 12-hydroxysteric acid such as Solutol® HS15 in a S/O weight ratio with the lipophilic component (e.g., a fish oil) of 1:1 to 5:1, and can be about 3:1 to 5:1 or about 5:1.

In another example, a nanoemulsion includes an imidazole or imidazole analog such as 5-(3,3-Dimethyl-1-triazenyl)imidazole-4-carboxamide (“dacarbazine”). Example 5 illustrates the preparation of such nanoemulsions using dacarbazine. A nanoemulsion can include the imidazole or imidazole analog analog in a fish oil, such as cod liver oil. When the nanoemulsion includes a polyoxyethylene ester of 12-hydroxysteric acid surfactant such as Solutol® HS15, the lipophilic component can be formulated without rice bran oil. The imidazole or imidazole analog nanoemulsion can include a polyoxyethylene ester of 12-hydroxysteric acid such as Solutol® HS15 with a fish oil lipophilic component in a S/O weight ratio of 1:1 to 5:1, and can be about 3:1 to 5:1 or about 5:1. Such nanoemulsions are useful, for example, for administration for the treatment of an antineoplastic chemotherapy drug used in the treatment of various cancers, such as malignant melanoma, Hodgkin lymphoma, sarcoma, and islet cell carcinoma of the pancreas.

Formulations including dacarbazine in rice bran oil as the lipophilic component, water and the polyoxyethylene ester of 12-hydroxysteric acid surfactant Solutol® HS15 did not form a stable nanoemulsion according to the method illustrated in FIG. 1, while comparable formulations substituting fish oil for the rice bran oil in the lipophilic component did form stable nanoemulsions by the same method. Therefore, the dacarbazine nanoemulsion can be at least substantially free of rice bran oil. Rice bran oil has a higher percentage (about 4%) of nonsaponifiable components than fish oil. The dacarbazine nanoemulsion can be formulated with oils that include omega 3 or omega 6 fatty acids. Formulations comprising dacarbazine can include a lipophilic component with oils characterized by nonsaponifiable components that are less than about 4%.

In another example, a nanoemulsion includes a taxane compound such as paclitaxel (CAS 33069-62-4). Example 6 illustrates the preparation of such nanoemulsions using paclitaxel. A nanoemulsion can include the taxane compound in a lipophilic component selected from the group consisting of soybean oil, coconut oil, cod liver oil or other fish oil, and/or rice bran oil. The pyrimidine or pyrimidine analog nanoemulsion can include a polyoxyethylene ester of 12-hydroxysteric acid such as Solutol® HS15 in a S/O weight ratio with the lipophilic component (e.g., rice bran oil) of 1:1 to 5:1, such as about 3:1 to 5:1 or about 4:1 to 5:1, or 5:1.

In another example, a nanoemulsion includes a tocopherol such as Vitamin E and/or a tocotrienol compound (including mixture of alpha-, beta-, gamma- and delta-tocotrienols) Example 7 illustrates the preparation of such nanoemulsions using a mixture of tocotrienol isomers. A nanoemulsion can include the tocopherol and/or a tocotrienol compound(s) in a rice bran oil. When the nanoemulsion includes a polyoxyethylene ester of 12-hydroxysteric acid surfactant such as Solutol® HS15, the lipophilic component can include rice bran oil. In particular, the tocopherol and/or a tocotrienol nanoemulsion can include a polyoxyethylene ester of 12-hydroxysteric acid such as Solutol® HS15 with a rice bran oil lipophilic component in an S/O weight ratio of 1:1 to 5:1, and/or about 3:1 to 5:1 or about 5:1. Such nanoemulsions are useful, for example, for administration as an antioxidant.

Formulations including tocotrienols in rice bran oil as the lipophilic component, water and the C20 ethoxylated monoglyceride such as EMG-20 did not form a stable nanoemulsion according to the method illustrated in FIG. 1, while comparable formulations substituting the polyoxyethylene ester of 12-hydroxysteric acid surfactant Solutol® HS15 for EMG-20 did form stable nanoemulsions by the same method. The tocopherol and/or a tocotrienol nanoemulsion, particularly a nanoemulsion containing rice bran oil, which can be at least substantially free of a C20 ethoxylated monoglyceride such as EMG-20.

In another example, a nanoemulsion includes a carotenoid (including a xanthophyl compound and/or a zeaxanthin compound), such as 4-[18-(4-Hydroxy-2,6,6-trimethyl-1-cyclohexenyl)-3,7,12,16-tetramethyloctadeca-1,3,5,7,9,11,13,15,17-nonaenyl]-3,5,5-trimethyl-cyclohex-2-en-1-ol (“lutein”). Example 8 illustrates the preparation of such nanoemulsions using lutein. A nanoemulsion can include the lutein in a rice bran oil as the lipophilic component. The lutein nanoemulsion can include a C20 ethoxylated monoglyceride such as EMG-20 with the rice bran oil lipophilic component in an S/O weight ratio of 5:1 to 1:1, and can be about 5:1 to 3:1 or about 5:1. Such nanoemulsions are useful, for example, for administration for the treatment of an antineoplastic chemotherapy drug used in a pharmaceutical, nutraceutical, human food or pet food formulations.

However, formulations including lutein in rice bran oil as the lipophilic component, water, and the C20 ethoxylated monoglyceride EMG-20 did not form a stable nanoemulsion according to the method illustrated in FIG. 1, while comparable formulations substituting rice bran oil for the soybean oil in the lipophilic component did form stable nanoemulsions by the same method. The lutein nanoemulsion can be at least substantially free of rice bran oil when the surfactant is the C20 ethoxylated monoglyceride EMG-20.

In another example, a nanoemulsion includes a polynucleotide or polypeptide compound (including siRNA compounds). The SANE compositions disclosed herein can comprise oligonucleotides for use in antisense modulation of the function of DNA or messenger RNA (mRNA) encoding a protein the modulation of which is desired, and ultimately to regulate the amount of such a protein. Hybridization of an antisense oligonucleotide with its mRNA target interferes with the normal role of mRNA and causes a modulation of its function in cells. The functions of mRNA to be interfered with include all vital functions such as translocation of the RNA to the site for protein translation, actual translation of protein from the RNA, splicing of the RNA to yield one or more mRNA species, turnover or degradation of the mRNA and possibly independent catalytic activity which can be engaged in by the RNA. The overall effect of such interference with mRNA function is modulation of the expression of a protein.

In the context of the present invention, “modulation” means either an increase (stimulation) or a decrease (inhibition) in the expression of a gene. In the context of the present invention, inhibition is one form of modulation of gene expression and mRNA is one target. SANE compositions comprising antisense compounds can be used as research reagents diagnostic aids, and therapeutic agents. For example, antisense oligonucleotides, which are able to inhibit gene expression with specificity, can be used to elucidate the function of particular genes. Antisense compounds are also used, for example, to distinguish between functions of various members of a biological pathway.

SANE compositions can also include other oligomeric antisense compounds, including, but not limited to, oligonucleotide mimetics. The antisense compounds can be active components in a SANE composition and have from about 8 to about 30 nucleotide bases (i.e., from about 8 to about 30 linked bases), although both longer and shorter sequences may find use with the present invention. For example, such antisense active components can be antisense oligonucleotides, such as those comprising from about 12 to about 25 nucleotides.

Nanoemulsions including small interfering RNA (siRNA) were prepared. siRNA can be used as sequence-selective inhibitors of transcription. Example 9 illustrates the preparation of such nanoemulsions using siRNA compounds that are sequence-selective inhibitors of transcription. A nanoemulsion can include the polynucleotide or polypeptide compound(s) in a lipophilic component such as gelatin. In particular, the polypeptide or polynucleotide nanoemulsion can include an ethoxylated monoglyceride (e.g., EMG-20) surfactant and/or a polyoxyethylene ester of 12-hydroxysteric acid surfactant (e.g., Solutol® ®HS15) with a gelatin lipophilic component in a S/O weight ratio of 5:1 to 1:1, including about 1:1. SANE compositions may include active components comprising nucleic acid molecules selectively screened to bind to a selected target. For example, screening can conducted using the technique known as SELEX. The basic SELEX procedure is described in U.S. Pat. Nos. 5,475,096 and 5,270,163 (herein incorporated by reference in their entireties). The SELEX procedure can allow for identification of nucleic acid molecules with unique sequences, each of which has the property of binding specifically to a desired target compound or molecule.

In another example, a nanoemulsion includes insulin. Example 10 illustrates the preparation of such nanoemulsions using insulin formulated for transdermal administration. A nanoemulsion can include the insulin in a lipophilic component such as soybean oil, a surfactant including a combination of Polysorbate 80 (also known as TWEEN® 80) and Sorbitan monooleate (Span 80) and water.

### Consumer Product SANE Compositions

The SANE compositions can be formulated in a variety of consumer products. In particular, SANE compositions consisting essentially of the lipophilic component, the hydrophilic component and the surfactant can be added to consumer products, without including additional active components within the SANE compositions (e.g., SANE compositions without pharmaceutical, nutraceutical, or cosmaceutical ingredients that are not required for, or involved in, the formation of the SANE composition). Such three component SANE compositions can be used to add the lipophilic component to the consumer product in a form that increases bioavailability, absorption, or effectiveness of the lipophilic component during its intended use. For example, the nanoemulsion of the lipophilic component may increase the rate of absorption of a fish oil supplement, increase the rate of permeation of a vitamin lipophilic component into the skin from a bandage or wound dressing, or increase the reactive surface area and desired reactivity of a lipophilic component in a cosmetic product applied to the skin or hair in a moisturizer or shampoo.

A SANE composition can include an aqueous component, an oil component, and a surfactant mixture component. In certain embodiments, the aqueous component is selected from distilled water, deionized water, normal saline, phosphate buffered saline and mixtures thereof. In particular embodiments, SANE compositions described herein are for inclusion in a cosmetic, nutritional or therapeutic formulation. Alternatively, formulations are provided comprising the SANE composition as a component in combination with other materials appropriate for such products.

The SANE composition can be combined with carriers used in consumer product preparations, such as ceteareth-20, ceteareth-12, glyceryl stearate, cetearyl alcohol and cetyl palmitate (e.g., from 1.0 to 2.5% by weight, based on the total weight of the composition of the formulation). Additional components of cosmetic formulations comprising the SANE composition can include Emulgin B2 (ceteareth-20) and Emulgade® SE. In certain embodiments, the aqueous component constitutes approximately 0.1-35% of the nanoemulsion formulation. In other embodiments, the aqueous component constitutes approximately 1-20% of the nanoemulsion formulation (e.g., the aqueous component constitutes approximately 2-10%, 2-5% or less than 6% of the nanoemulsion formulation).

In another example, the SANE composition is formulated with or as filmogenic agents, emollients, solvents, and/or skin conditioners. A few examples of silicone that can be added to or combined with a SANE composition include volatile and non-volatile silicone oils such as, for example, cyclomethicone, alkyldimethicones, dimethicone-copolyols, dimeticonols, phenyl trimethicones, caprylyl trimethicones, aminofunctional silicones, phenyl modified silicones, phenyl trimethicones, alkyl modified silicones, dimethyl and diethyl polysyloxane, C1-C30 mixed alkyl polysiloxane, α-methyl-ω-methoxypolymethylsiloxane, polyoxydimethylsililene, polydimethyl silicone oil and combinations thereof, or silicone elastomers such as cyclomethicone crosspolymer and dimethicone, vinyl dimethicone crosspolymer and dimethicone, dimethicone crosspolymer and dimethicone and cyclopentasiloxane crosspolymer and dimethicone.

In another example, the SANE composition is formulated with or as an emollient composition. The function of emollients in cosmetic compositions is to add or replace lipids and natural oils to the skin. As emollients to be added to or combined with an existing SANE composition, one can use conventional lipids such as, for example, oils, waxes, and other water-soluble components and polar lipids that are modified lipids so as to increase their solubility in water by esterification of a lipid to a hydrophilic unit such as, for example, hydroxyl, carbonyl groups, among others. Some compounds that can be used as emollients are natural oils such as essential oils and plant derivatives, esters, silicone oils, polyunsaturated fatty acids, lanoline and derivatives thereof. Some natural oils that can be used are derived from damson, passion fruit, Para-nut, carap nut, cupuassu, sesame, soybean, peanut, coconut, olive, cocoa, almond, avocado, carnauba, cotton seed, rice bran, peach stone, mango stone, jojoba, macadamia, coffee, grape seed, pumpkin seed, among others, and mixtures thereof. In addition, a number of natural compounds can be used, as for example, microcrystalline wax, carnauba wax, Shea butter, bee-wax, ozokeri wax, among others and mixtures of waxes and/or oils.

In another example, the SANE compositions are formulated with or as a consumer product such as a moisturizing and/or wetting agent. The moisturizing agent can be formulated with a SANE composition to promote the retention of water in the skin (e.g., a composition capable of supplying water to the skin and/or preventing the loss of water from the skin). The wetting agent further helps in increasing the efficacy of the emollient, reduces skin peeling and improves the sensorial properties of the skin (softness, smoothness). A few examples of wetting agents that can be added to or combined with the nanoemulsion of the present invention are: glycerin, glycereth-26, PET-4 dilaurate, polyhydroxyl alcohols, alkylene polyols and derivatives thereof, glycerol, ethoxylated glycerol, propoxylated glycerol, sorbitol, hydroxypropyl sorbitol, among others, lactic acid and lactate salts, diols and C3-C 6 triols, Aloe vera extract in any form, as for example, in the form of a gel, sugars, and starches and derivatives thereof, as for example, alkoxylated glucose, hyaluronic acid glycolic acid, lactic acid, glycolic acid and salicylic acid, pantenol and urea. Optionally, a SANE composition can be combined with or can include a perfume or fragrance selected from any suitable substances. For example, the nanoemulsion can be formulated for incorporation into a consumer product such as an antiperspirant formulation or laundry detergent.

Consumer product formulations comprising a SANE composition can further include lipophilic or hydrophilic components used for similar cosmetic applications. These other components can include, for example, seaweeds, a combination of palmitoyl hydroxypropyl trimonium aminopectin, glycerin crosspolymer, lecithin and grape-seed extract, bisabolol (anti-inflammatory active), D-pantenol (conditioning active), tocopherol (vitamin E), retinol (vitamin A), ascorbic acid (vitamin C), erocalcipherol (vitamin D) and sunscreen commonly added to compositions of products for topical or hair use; dyes; chelating agents as ethylenediaminotetraacetic acid (EDTA) and salts thereof; pH adjusting agents, like triethanolamine; preservatives like DMDM hydantoin; plant extracts such as chamomile, rosemary, thyme, calendula, carrot extract, common juniper extract, gentian extract, cucumber extract; skin conditioning agents; lipophilic substances; antioxidant agents, like butyl hydroxytoluene (BHT), butyl hydroxyanisol (BHA); and other commercially accepted components which are compatible with the base composition comprising the SANE composition.

Other examples of formulations comprising the SANE composition include nutritional supplements, beverages, nutrient bars and other foods, moisturizers, sunscreens, shampoos, cosmetic products, injectable bulking agents, and toothpastes. In one example, the SANE composition can be formulated as or added to cosmetic formulations for care, protection and makeup of skin, mucosa, scalp and hair. The nanoemulsion produced according to production methods described herein can be used as final product for application over the skin, mucosa and hair, or can also be incorporated in previously prepared cosmetic compositions, acting as an additive.

In one embodiment, the present invention provides SANE compositions including a chemotherapeutic active component and methods to deliver chemotherapeutic compounds (i.e., dacarbazine) to primary breast cancer tumors and metastases, which provide more effective absorption of the chemotherapeutic compounds into a tumor cell than the administration of the active component without a nanoemulsion. The SANE compositions can be formulated for dermal (e.g., a non-toxic oil) having an average droplet size of less than 50 nm or 25 nm can be administered systemically, transdermally, or by injection a vitamin oil nanoemulsion formulated in a shaving cream or aftershave composition) or transdermal administration (e.g., a vitamin oil nanoemulsion in a patch or bandage).

In another example, the SANE composition can be formulated with lipophilic nanoparticles or droplets in water (e.g., for addition to a beverage such as juices, e.g., orange, grape, apple, cherry, mango, peach, blueberry, or pomegranate juice, or a carbonated or non-carbonated soft drink or other beverage). In yet another example, the SANE composition can be formulated for incorporation into a sun screen.

In some embodiments, the components of the SANE compositions and desired active components agents can be separated into individual formulations (e.g., individual vials) for later mixing during use, as can be desired for a particular application. Such components can advantageously be placed in kits for diagnostic or therapeutic use. In some embodiment, such kits contain all the essential materials and reagents required for the delivery of biological agents via the nanoemulsion formulations of the present invention to the site of their intended action.

### Pharmaceutical SANE Compositions

SANE compositions comprising one or more active components can be formulated in a therapeutically effective and medically appropriate manner for use in methods of treatment. The therapeutically effective dose of an active component formulated in a SANE composition can be less than the therapeutically effective dose of the active agent that is not administered as a nanoemulsion.

A SANE composition can also serve as carriers for one or more active components. Such active components can be added prior to preparing the nanoemulsion to the hydrophilic phase or to the lipophilic phase. Active components can be made to attach to oil particles and/or are incorporated and/or dissolved therein. SANE compositions containing active components can be utilized for the production of pharmaceutical, and/or nutraceutical preparations where the nanoemulsion is mixed, as the active component, with a solid or liquid vehicle suitable for therapeutic administration. The mixture of the SANE composition comprising the active component and other carrier components can be formulated and provided as ampoules, especially sterile injection and infusion solutions; solutions (e.g., oral liquids, eye drops and nose drops which can contain various substances in addition to the nanoemulsion); aerosols and dosing aerosols (e.g., further including a propellant gas and/or stabilizers besides the nanoemulsion); hydrophilic and hydrophobic gels and ointments containing the nanoemulsion; o/w or w/o creams containing the nanoemulsion; lotions and pastes containing the nanoemulsion.

Nanoemulsions produced in accordance with the process described herein can also be utilized with advantage for the preparation of nutrient solutions for cell cultures by adding to the nanoemulsions, for example, natural amino acids, antibiotics, small amounts of transferrin and optionally glucose. In such nutrient solutions, the nanoemulsions serve as energy deliverers and can at least in part replace the proteins used in conventional nutrient solutions, for example those made from calf serum. SANE compositions including an active component (e.g., encapsulated in droplets of the lipophilic component with an average particle size of less than 100, 50 or 25 nm) can be formulated for, and administered using, any medically appropriate route of administration including, but not limited to, oral, transdermal, intravenous, intraperitoneal, intramuscular, intratumoral, or subcutaneous routes. For example, administration of a uniform SANE composition comprising one or more active components can intracellularly deliver chemotherapeutic compounds to target cells (e.g., metastasized tumor cells within the subject), or provide improved membrane permeability properties (e.g., during transdermal delivery) deliver the active component into a subject.

Formulations including SANE compositions disclosed herein can further comprise other supplementary biological agents such as pharmaceutically acceptable carriers, or diluents. Examples of pharmaceutically acceptable carriers include, but are not limited to, a liquid, cream, foam, lotion, or gel, and can additionally comprise organic solvents, emulsifiers, gelling agents, moisturizers, stabilizers, wetting agents, preservatives, time release agents, and minor amounts of humectants, sequestering agents, dyes, perfumes, and other components commonly employed in pharmaceutical compositions.

In one embodiment, the present disclosure provides SANE compositions include a chemotherapeutic active component and methods to deliver chemotherapeutic compounds (i.e., dacarbazine) to primary breast cancer tumors and metastases, which provide more effective absorption of the chemotherapeutic compounds into a tumor cell than the administration of the active component without a nanoemulsion. A SANE composition comprising at least one chemotherapeutic compound (i.e., for example, dacarbazine and/or tamoxifen) within droplets or particles of a lipophilic component (e.g., a non-toxic oil) having an average droplet size of less than 50 nm or 25 nm can be administered systemically, transdermally, or by injection.

### Administration of SANE Compositions

The nanoemulsion formulations of the present invention can administered in any acceptable manner. In some embodiments, the nanoemulsion formulations of the present invention are delivered to a subject by parenteral administration. Parenteral administration includes, but is not limited to, administration intravenously, intra-muscularly, subcutaneously, intradermally, intraperitoneally, intrapleurally, or intrathecally.

In some embodiments, the SANE formulations described herein are delivered to a subject by non-parenteral routes of administration. Non-parenteral administration refers to the administration, directly or otherwise, of the nanoemulsion formulations of the present invention via a non-invasive procedure which typically does not entail the use of a syringe and needle. Non-parenteral administration includes, but is not limited to, the contacting, directly or otherwise, to all or a portion of the alimentary canal, skin, eyes, pulmonary tract, urethra or vagina of an animal. Specific examples of non-parenteral administration, include, but are not limited to, buccal, sublingual, endoscopic, oral, rectal, transdermal, nasal, intratracheal, pulmonary, urethral, vaginal, ocular, and topical.

The SANE formulations described herein can be delivered to a subject via the alimentary canal, the tubular passage in animal that functions in the digestion and absorption of food and the elimination of food residue, which runs from the mouth to the anus, and any and all of its portions or segments (e.g. the oral cavity, the esophagus, the stomach, the small and large intestines and the colon, as well as compound portions thereof like the gastro-intestinal tract). Therefore, delivery to the alimentary canal encompasses several routes of administration including, but not limited to, oral, rectal, endoscopic and sublingual/buccal administration.

In some embodiments, the non-parenteral administration of the SANE formulations described herein can include iontophoresis (the transfer of ionic solutes through biological membranes under the influence of an electric field), phonophoresis or sonophoresis (use of ultrasound to enhance the absorption of various therapeutic agents across biological membrane, notably the skin and cornea). These techniques can be used to enhance the transport of the nanoemulsion formulations of the present invention such that biological agents in the nanoemulsion formulations are able to have a therapeutic effect.

Delivery of the SANE compositions described herein can occur via the oral mucosa, as in the case of buccal and sublingual administration. These routes of administration can have several desirable features, including, in many instances, a more rapid rise in plasma concentrations of the biological agents, than via oral delivery. Furthermore, because venous drainage from the mouth is to the superior vena cava, this route also bypasses rapid first-pass metabolism by the liver.

Endoscopy can be used for delivery of the SANE compositions described herein directly to an interior portion of the alimentary tract. For example, endoscopic retrograde cystopancreatography (ERCP) takes advantage of extended gastroscopy and permits selective access to the biliary tract and the pancreatic duct. The nanoemulsion formulations of the present invention can be delivered directly into portions of the alimentary canal (e.g. duodenum or gastric submucosa) via endoscopic means. Gastric lavage devices and percutaneous endoscopic feeding devices can also be used for direct alimentary canal delivery of the SANE compositions.

The SANE compositions described herein can be administered by a lower enteral route (e.g., through the anus into the rectum or lower intestine). Rectal suppositories, retention enemas or rectal catheters can be used for this purposed and can be used (e.g. pediatric, geriatric, or unconscious patients).

The SANE compositions described herein are delivered topically (locally) to a subject. Topical application of the nanoemulsion formulations primarily produces local effects. Examples of topical application include, but are not limited to, topical application to mucous membranes, skin, eyes, or to organ surfaces (either ex vivo transplant organs or in vivo organs). One topical route of administration is through the skin. Topical delivery of the SANE compositions disclosed herein can have the advantage of directing the biological agents in the nanoemulsion formulations to the confined site of disease (e.g. clinically active skin lesions). Topical application of the nanoemulsion formulations of the present invention can be, for example, in the form of a transdermal patch, impregnated into absorptive materials, such as sutures, bandages, and gauze, or coated onto the surface of solid phase materials. The SANE compositions disclosed herein can be administered in formulations that are non-irritating to the skin of a subject.

The SANE compositions described herein can be delivered through mucous membranes. Nanoemulsion formulations applied to mucous membranes can be formulated to provide primarily local effects. This route of administration includes application of the nanoemulsion formulations to mucous membranes of the conjunctiva, nasopharynx, oropharynx, vagina, colon, urethra, and urinary bladder. Ocular delivery of the SANE compositions described herein is useful for the local treatment of eye infections or abnormalities. The nanoemulsion formulation can be administered via instillation and absorption occurs through the cornea. Corneal infection or trauma can thus result in more rapid absorption.

In some embodiments, the components of the SANE compositions and desired active components agents can be separated into individual formulations (e.g., individual vials) for later mixing during use, as can be desired for a particular application. Such components can advantageously be placed in kits for diagnostic or therapeutic use. In some embodiment, such kits contain all the essential materials and reagents required for the delivery of biological agents via the nanoemulsion formulations of the present invention to the site of their intended action. In some embodiments, the kits comprise fully assembled formulations.

The kits can also include a means for containing the vials in close confinement for commercial sale (e.g., injection or blow-molded plastic containers into which the desired vials are retained). Irrespective of the number or type of containers, the kits of the invention also can comprise, or be packaged with, an instrument for assisting with the administration or placement of the nanoemulsion formulation on or in a subject. Examples of such instruments include, but are not limited to, inhalers, syringes, pipettes, forceps, measured spoons, eye-droppers, swabs, patches, or any such medically approved delivery vehicle.

### EXAMPLES

The invention is further described in the following examples, which do not limit the scope of the invention described in the claims. Methods of making, analyzing, and characterizing some aspects of the nanoemulsions are described below.

Rice bran oil, unless otherwise indicated, contains poly unsaturated (5.5 gms), mono unsaturated (6 grams) and saturated fats (2.5 grams) out of 14 grams of fat available for 1TBSP. Fish oil, unless otherwise indicated, contains cod liver oil with 4.6 grams having 7% total fat (including 0.5 grams, 3% saturated fat, 1.6 g polyunsaturated fat and 2.5 grams monounsaturated fat), 25 mg (8%) cholesterol, 500 mg EPA and 500 mg DHA. The surfactant Solutol® HS15 is a tradename (BASF Aktiengesellschaft) for a Polyethylene glycol 660 hydroxystearate as a nonionic solubilizer for injection. Unless otherwise indicated, EMG 20 is Ethoxylated Mono- and Diglycerides obtained from Caravan Ingredients (Lenexa, Kans.) and TWEEN® 80 and CREMOPHOR surfactants were obtained from Sigma. The CREMOPHOR can be the product CREMOPHOR EL®, which is a registered trademark of BASF Corp. for its version of polyethoxylated castor oil. It is prepared by reacting 35 moles of ethylene oxide with each mole of castor oil.

In contrast to some nanoemulsions formed using microfluidization techniques (i.e., high pressure emulsion forming techniques which can produce emulsions with particle or droplet size distributions having multiple peaks), the SANE compositions can have a single peak distribution of droplet sizes of the lipophilic component suspended in the hydrophilic component, such as a single peak particle size distribution with an average size (Z-avg) of up to about 25 nm. Unless otherwise indicated, a “single peak particle size distribution” refers to an emulsion where a histogram graph of measured particle or droplet sizes has a single, as opposed to multiple, peaks.

Particle sizes of all the formulations indicated in the Examples below were measured by dynamic laser light scattering, also called Photon correlation spectroscopy, using the Malvern Zetasizer-S instrument (Malvern Instruments Inc., Southborough Mass.). Each sample was diluted immediately before measurement with distilled water to avoid multiple light scattering effects. A previous report has indicated that the dilution of samples did not change the particle size distribution (Muller et al., 2002). The particle diffusion (translational diffusion) due to Brownian motion was measured in the instrument and related to the size of the parameter. The mean hydrodynamic diameter (DH) was calculated from the Strokes-Einstein equation. The range of particle sizes which can be measured by the Zetasizer is from 0.6 to 6000 nm.

The in vitro activity of several nanoemulsions described in the Examples were tested with CCL-221, Malme 3M and/or CCL-2 cancer cell lines. In each such Example, the human cultured cancer cell line CCL-221, Malme 3M and CCL-2 were obtained from the American Type Culture Collection (ATCC) (Manassas, Va.). CCL-221 is colorectal adenocarcinoma cell line which was isolated by D. L. Dexter and associates during a period from 1977-1979. CCL-221 was cultured in ATCC-formulated RPMI-1640 medium with fetal bovine serum to a final concentration of 10%. Malme-3M, a malignant melanoma derived from a 43 year old male was cultured in Iscove's Modified Dulbecco's Medium (IMDM) and supplemented with 10% fetal bovine serum. CCL-2, a Cervix epithelial adenocarcinoma cell line from 31 year old female, was cultured in Dulbecco's Modified Eagle's medium (DMEM) with 10% fetal bovine serum. All media were also supplemented with 100 unites penicillin, 100 μg streptomycin per ml, and 1 mM sodium pyruvate. Cells were cultured at 37 degrees C. in a 95% O2: 5% CO2 incubator after subculture as indicated by the manufacture.

### Example 1

### Compositions and Methods for Producing Self-Assembled Nanoemulsions without Additional Active Components

A series of nanoemulsions without an active component (“blank nanoemulsions”) were prepared with various oil and different surfactant concentrations. Briefly, 1 g (2 wt %) of oils including soybean oil, coconut oil, fish oil, and rice bran oil were weighed and placed in the beakers, respectively. Subsequently, 1 g (2 wt %), 3 g (6 wt %), 5 g (10 wt %), 7 g (14 wt %) of one of the following ethoxylated non-ionic surfactant was added to each composition: a C20 ethoxylated monoglyceride (EMG-20), polyoxyethylene ester of 12-hydroxysteric acid (Solutol® HS15), Polyoxyethylene Sorbitan Monooleate (TWEEN® 80), and polyoxyethylene castor oil, for a total of 64 nanoemulsions. The surfactant was added to the oil and the mixture was heated and stirred for 5 minutes using a magnetic stirrer until the two components visually formed a homogeneous mixture and the temperature at this time was 50-60 degrees C. Distilled water (total volume=50 mL) was then added while the mixture was stirring at 60 degrees C. At this stage it forms an O/W emulsion. During heating, when the PIT (or HLB temperature) of the system reached (65-70 degrees C., phase inversion zone), the surfactant was in equilibrium with the oil and water phase. Heating and stirring was continued beyond the PIT up to 80 degrees C. At this temperature the system inverts to a W/O emulsion. The emulsion was cooled at RT to obtain an O/W emulsion.

Blank nanoemulsions were prepared using several different types of oils including soybean oil, coconut oil, rice bran oil, and fish oil, and mixed with Solutol® HS15 in different initial weight ratios of Surfactant/Oil (S/O ratio). Measurements of the average oil droplet (“particle”) size in each nanoemulsion are shown in FIGS. 2-5. The data shows that particle size was dramatically affected by the S/O ratio, with dramatic reductions in the average oil droplet size when the S/O ratio is increased from 1:1 to 3:1, with additional average oil droplet size reductions achieved by increasing the initial S/O ratio above 3:1 to 5:1 an 7:1 (FIGS. 2-5). Accordingly, multiple nanoemulsions having average oil droplet sizes below 25 nm were prepared, including (a) nanoemulsions of rice bran oil in water using a 5:1 S/O initial weight ratio with EMG20, Solutol® HS15 or CREMOPHOR EL® surfactants (FIG. 2); (b) nanoemulsions of coconut oil in water using a 5:1 S/O initial weight ratio with EMG20, Solutol® HS15, TWEEN® 80 or CREMOPHOR EL® surfactants (FIG. 3); (c) nanoemulsions of soybean oil in water using a 5:1 S/O initial weight ratio with EMG20, Solutol® HS15, TWEEN® 80 or Cremophor surfactants (FIG. 4); and (d) nanoemulsions of fish oil in water using a 5:1 S/O initial weight ratio with EMG20, Solutol® HS15, or TWEEN® 80 surfactants (FIG. 5).

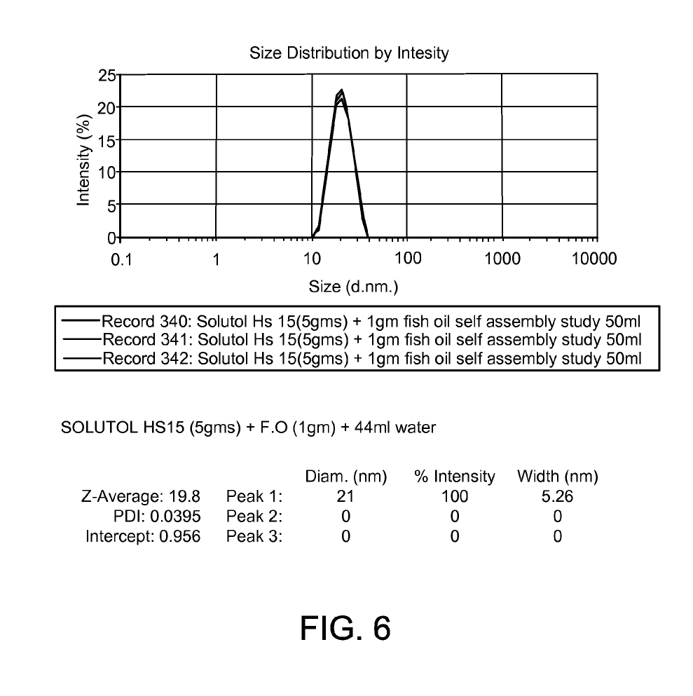

The formation of nanoemulsion exhibited high monodispersity and stability as S/O ratio equal to 5:1. The particle size of blank nanoemulsion formulated with omega-3 fish oil in an S/O ratio of 5:1 was approximately 20 nm and particle size distribution having a width of less than 10 nm (i.e., an average droplet size of about 19.8 nm with a width about 5.26 nm) is shown in the graph of FIG. 6.

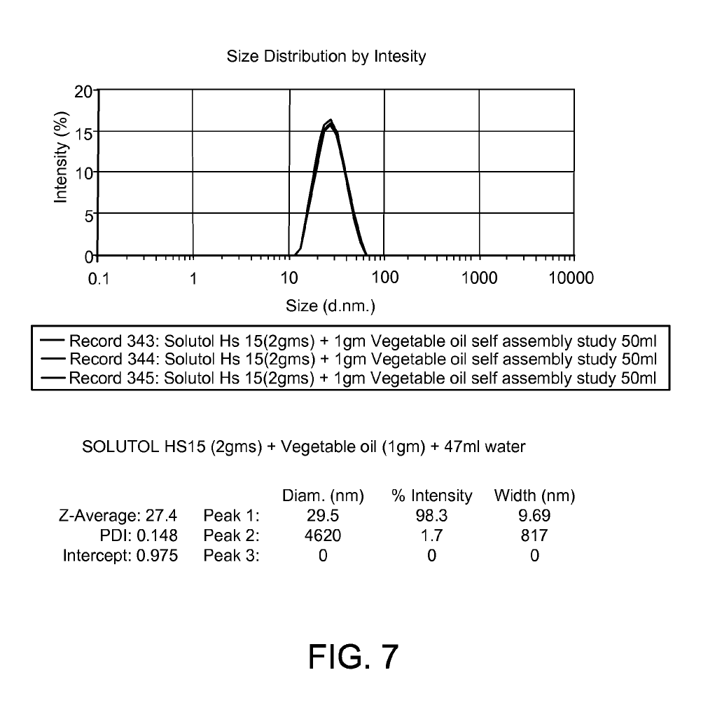

FIG. 7 shows the droplet particle distribution for a blank nanoemulsion formed by the self-assembly method in the manner described in Example 1 using vegetable oil and Solutol® SH15 surfactant at a 2:1 initial S/O weight ratio (2 grams Solutol® HS15 surfactant, 1 gram vegetable oil, and 47 mL water). The oil droplet particle size distribution has an average size of about 27 nm and a width of less than 10 nm (Z-average particle size of 27.4 nm, particle size peak width of 9.69 nm). Various weight ratios for each SANE formulation are tabulated in Table 1. Table 1 below summarizes four SANE formulations prepared according the Examples with different types of oil (soybean oil, coconut oil, fish oil, and rice bran oil) and different nonionic polyethoxylated surfactants (EMG-20, Solutol®HS15, TWEEN® 80, and Cremophor). The average droplet size of the oil in the resulting SANE composition is shown in FIGS. 2-5.

TABLE 1

| | Approx. | | | | | | | | | |
|---|---|---|---|---|---|---|---|---|---|---|
| | Water | Oil | | S/O | W/(W + O) | O/(W + O) | O/(O + S) | W/(W + S) | | |
| Compo- | Weight | Weight | Surfactant | weight | weight | weight | weight | weight | S/(S + W + O) | Water |
| sition | (g) | (g) | Weight (g) | ratio | ratio | ratio | ratio | ratio | weight % | wt % |
| 1 | 48.0 | 1.00 | 1.00 | 1.00 | 0.980 | 0.020 | 0.500 | 0.020 | 2.0% | 96.0% |
| 2 | 46.0 | 1.00 | 3.00 | 3.00 | 0.979 | 0.021 | 0.250 | 0.061 | 6.0% | 92.0% |
| 3 | 44.0 | 1.00 | 5.00 | 5.00 | 0.978 | 0.022 | 0.167 | 0.102 | 10.0% | 88.0% |
| 4 | 42.0 | 1.00 | 7.00 | 7.00 | 0.977 | 0.023 | 0.125 | 0.143 | 14.0% | 84.0% |

### Example 2

### SANE Compositions Including Coumarin

Nanoemulsions including coumarin (benzopyrone, CAS 91-64-5) as an active component were prepared by the self-assembly methods described in Example 1. The coumarin active component was combined with an oil lipophilic component as described below. Coumarin can be used, for example, as a rodenticide, a precursor for several anticoagulants (e.g., warfarin), and as a gain medium in some dye lasers.

A series of 0.01 mM coumarin nanoemulsions were prepared by first providing 1 g (2 wt %) of an oil selected from the group consisting of soybean oil, coconut oil, fish oil, and rice bran oil, weighed, and placed in a beaker. Coumarin was added into each beaker and heat and stirring were provided until the coumarin visually appeared to be dissolved in the oil. Subsequently, 5 g (10 wt %) of ethoxylated non-ionic surfactant, Solutol® HS15 (poly-oxyethylene esters of 12-hydroxystearic acid) was added and the mixture was heated and stirred for 5 mins using a magnetic stirrer, until the two components visually formed a homogeneous mixture and the temperature at this time was at 50-60 degrees C. Distilled water (total volume=50 mL) was then added while the mixture was stirring at 60 degrees C. At this stage it formed an O/W emulsion. During heating, when the PIT (or HLB temperature) of the system reached (65-70 degrees C., phase inversion zone), the surfactant was in equilibrium with the oil and water phase. Heating and stirring was continued beyond the PIT of the surfactant, up to 80 degrees C. At this temperature the system inverted to a W/O emulsion. The emulsion was cooled at RT to obtain a self-assembled O/W nanoemulsion.

In this study, the coumarin cell uptake was found to be significantly higher in the nanoemulsions bearing fish oil treated cells. Higher concentrations of omega-3 fatty acid can enhance absorption, bioavailability, and brain uptake following administration in the nanoemulsion formulation. A nanoemulsion made with polyunsaturated fatty acid (omega-3 and 6) can improve oral bioavailability and efficient brain delivery. CCL-221, Malme-3M, and CCL-2 were cultured in the appropriate cell media, grown to confluency and the nanoemulsions containing coumarin were added into a culture media at a ratio 1:50 and further incubated for 3 hours. After incubation, the cell media were removed by aspiration and PBS was used to wash the cells 3 times. After washing, 200 μL RIPA (lysis buffer) was added, and the cells were then harvested from the plate. 1 mL PBS will be used to collect the cells into the eppendorf. The collected cells were subjected to a fluorescence spectrophotometer to measure the fluorescence intensity of each nanoemulsion treated cells.

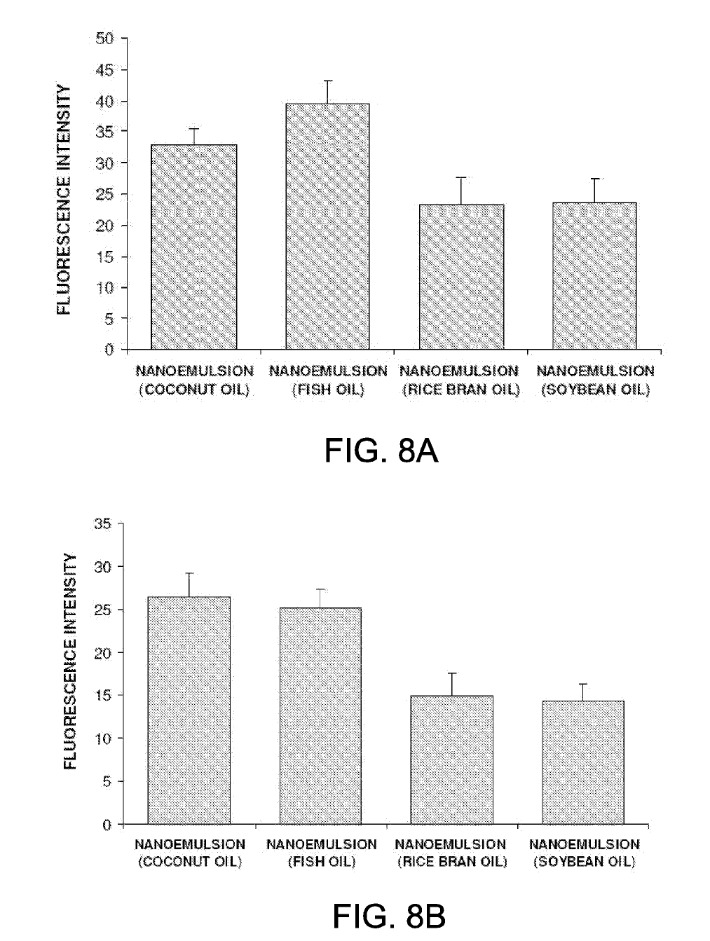

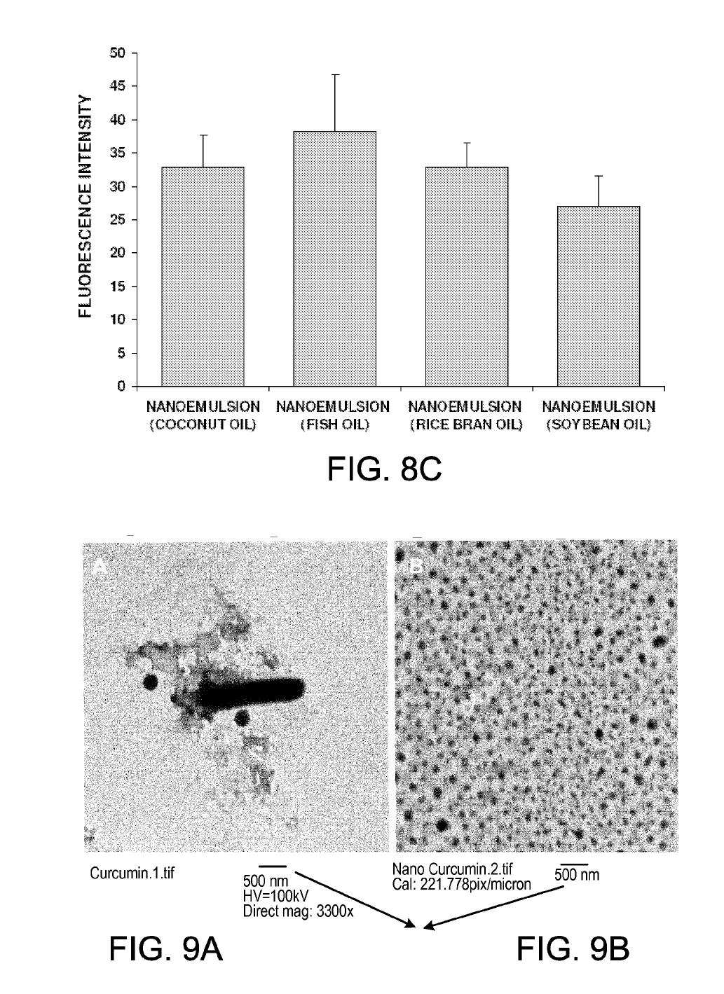

FIG. 8A is a graph showing the effect of different oils formulated via PIT nanoemulsion on colon cancer (CCL-221) cell uptake; FIG. 8B is a graph showing the effect of different oils formulated via PIT nanoemulsion on melanoma cancer (Melma-3M) cell uptake; FIG. 8C is a graph showing the effect of different oils formulated via PIT nanoemulsion on cervical cancer (CCL-2) cell uptake. These results showed that the tumor cell cultures (CCL-221, Malme-3M, and CCL-2) exhibited a similar pattern of coumarin uptake in different oil formulated nanoemulsion. In CCL-221 and Malme-3M tumor cell culture studies, the cell uptake of coumarin was relatively low (approximately 40% less) in the nanoemulsions bearing soybean oil and rice bran oil treated cells as compared to the cells treated with nanoemulsions bearing fish oil and coconut oil (FIGS. 8A and 8B).

As to the CCL-2 tumor cell culture experiment, the result showed that the cell uptake of coumarin was also lower in the nanoemulsions bearing soybean oil and rice bran oil treated cells (FIG. 8C). Therefore, in one example, SANE compositions for uptake by cells can be formulated using a lipophilic component that includes fish oil or coconut oil.

In addition, the following compositions were prepared: (1) SANE from a mixture of rice bran oil (1 gms), dextran (25 mg)+SOLUTOL HS15 surfactant (5 gm) deionized water (44 ml) and Coumarin 6 (11 mg) (formulation 1), (2) SANE from a mixture of rice bran oil (1 gms) and SOLUTOLHS15 surfactant (5 gm) deionized water (44 ml) and Coumarin 6 (11 mg) (formulation 2), and (3) a mixture of Coumarin 6 in regular solvent DMSO. Formulations 1 and 2 were nanoemulsions prepared by:

1. heating the rice bran oil (1 gm) and the dextran (25 mg) mix and then add Coumarin 6 (11 mg);
2. mix mixture while heating to form a solution of rice bran oil, dextran and coumarin 6 while heating;
3. add 5 gm of Solutol® HS15 and mix while heating;
4. add 44 ml of water and mix very well while heating above PIT; and
5. bring back to room temperature to form nanoemulsion.

The fluorescence of formulations 1-3 was measured (fluorescent spectrum. Abs/EM=458/503 nm). The relative fluorescence intensities of coumarin 6 in these formulations: 822 (formulation 3), 1108 (formulation 2), and 2448 (formulation 3). This data is shown in FIG. 24. Incorporation of dextran with a molecular weight of 1500 produced a nanoemulsion with an average particle size of 25 nm. FIG. 25 shows the increase in fluorescence intensity of the fluorescent dye Coumarin 6. Table 2 provides data showing the stability of the Coumarin preparations. Fluorescent intensity increased by 35% by making a nano-emulsion of Coumarin 6. Fluorescent intensity increased by 198% by making a nano-emulsion of Coumarin 6 with Dextran polymer. By adding polymer dextran we have increased the fluorescent intensity by 121% compared to nanoemulsion Coumarin 6 without Dextran.

TABLE 2
Stability of Coumarin preparations over several days stored at room temperature.

| Time | DMSO COUMARIN | | NANO-COUMARIN | | NANOCOUMARIN(DEXTRAN) | |
|---|---|---|---|---|---|---|
| (hrs) | Size | PDI | Size | PDI | Size | PDI |
| 0 | 1373 | 0.457 | 20 | 0.127 | 21 | 0.085 |
| 72 | 1373 | 0.857 | 20 | 0.123 | 21 | 0.085 |
| 96 | 2142 | 0.857 | 20 | 0.1 | 21 | 101 |
| 120 | 6000 | 0.857 | 20 | 0.1 | 22 | 0.175 |
| 144 | 6000 | 0.857 | 28 | 0.278 | 22 | 0.175 |
| 240 | 6000 | 0.857 | 28 | 0.278 | 22 | 0.175 |

PDI = polydispersity index

### Example 3

### SANE Compositions Including Curcumin

Self-assembled nanoemulsions including the polyphenol compound curcumin (i.e., (1E,6E)-1,7-bis(4-hydroxy-3-methoxyphenyl)-1,6-heptadiene-3,5-dione, CAS 458-37-7) were prepared with an average oil droplet particle size of less than 20 nm and a particle size distribution width of less than 5 nm. Curcumin is a nutraceutical with reported anti-cancer properties that inhibits cell proliferation in a melanoma cancer cell line. Curcumin was weighed out and mixed with 1 g mixture of fish oil and α-tocopherol as a ratio of 7:3 and 5 g Cremophor EL was added into the solution (i.e., a surfactant:oil ratio of 5:1). The mixture of the solution is then stirred and heated to 65 degrees C. Water (44 ml) was then added a few drops at a time until the solution eventually becomes clear. The curcumin SANE composition was formed by heating above the PIT of the surfactant for a time sufficient to invert the mixture from an O/W to a W/O emulsion, which was cooled below room temperature to form a nanoemulsion. The resulting SANE composition included curcumin encapsulated in the lipophilic component forming droplets suspended in water. The droplet particle sizes had a tightly defined distribution peak with a size of approximately 20 nm.

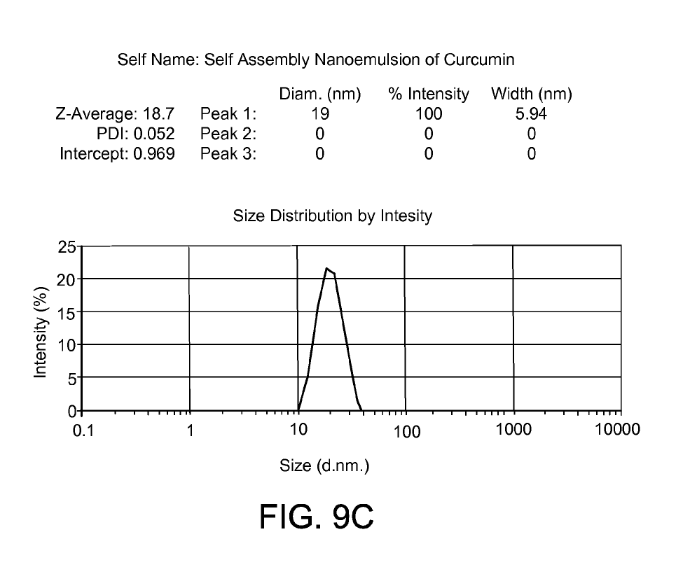

For comparison, a mixture of dimethyl sulfoxide (DMSO), water, and curcumin (i.e., not a nanoemulsion) was also prepared. FIGS. 9A and 9B show two transmission electron micrographs: (A) a TEM image of the DMSO prep of Curcumin (note clumping and irregular disorganized structures) and (B) the nanoemulsion (SANE) preparation of curcumin (note small particle size of about 20 nm and homogeneity of population). Both TEM images in FIG. 8 were characterized by a 182.093 pix/micron resolution and a solid bar corresponding to a scale of 500 nm in each image. FIG. 9C shows the oil/curcumin droplet size for the SANE composition, having an average droplet particle size of about 18.7 nm and a peak width of about 4.94 nm with a PDI of 0.052.

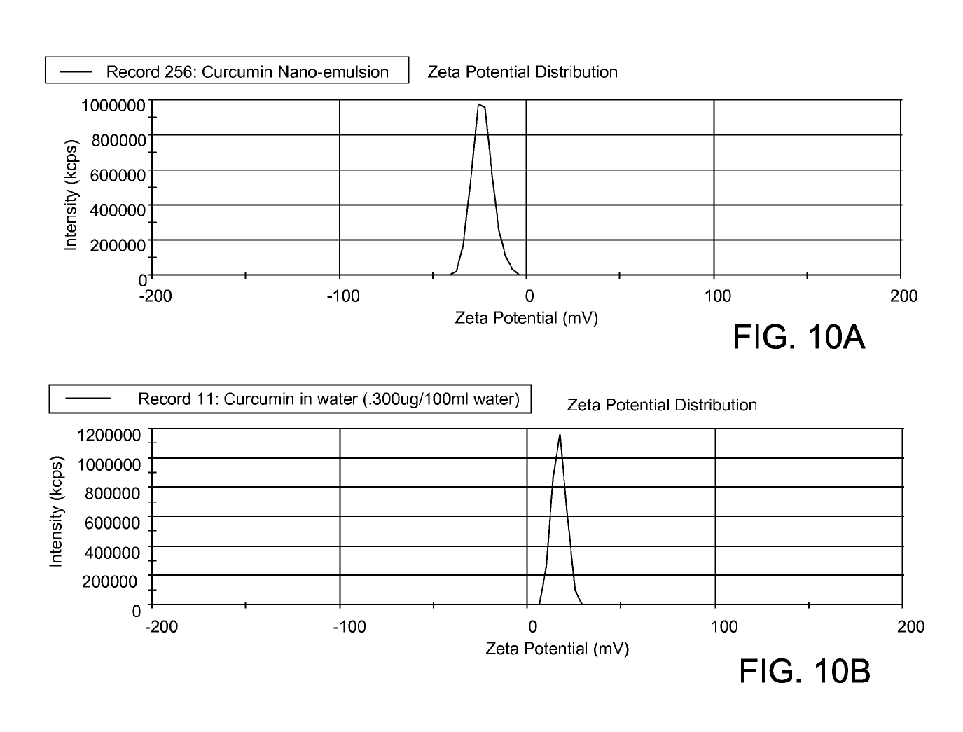

The electrokinetic potential (“Zeta-potential”) of the curcumin nanoemulsion was measured (FIG. 10A) and compared to the corresponding electrokinetic potential for curcumin in water (FIG. 10B). The zeta potential of a colloidal system corresponds to the electric potential in the interfacial double layer (DL) at the location of the slipping plane versus a point in the bulk fluid away from the interface (i.e., the potential difference between the dispersion medium and the stationary layer of fluid attached to the dispersed particle). For small particles (e.g., nanoemulsions) a higher zeta potential corresponds to greater stability of a colloidal dispersion, representing a greater degree of repulsion between adjacent particles in the dispersion to resist particle/droplet aggregation. A zeta potential of −23.5 (wall zeta potential of −50.3) was measured for the curcumin nanoemulsion (FIG. 10A), compared to a zeta wall potential at 10 mv of −2.53 for curcumin in water (FIG. 10B).

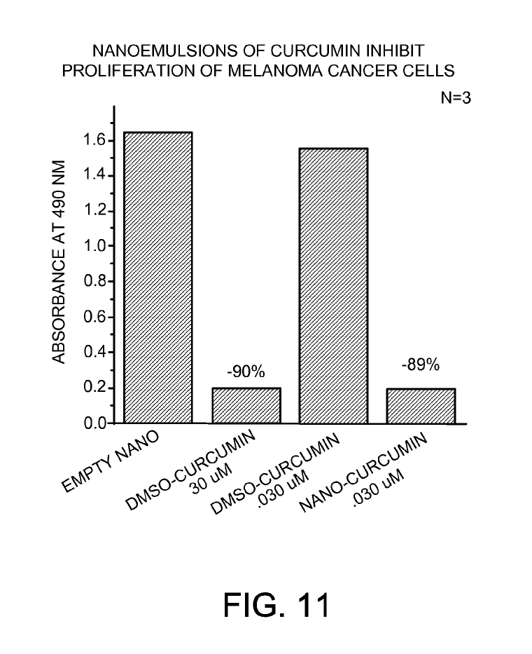

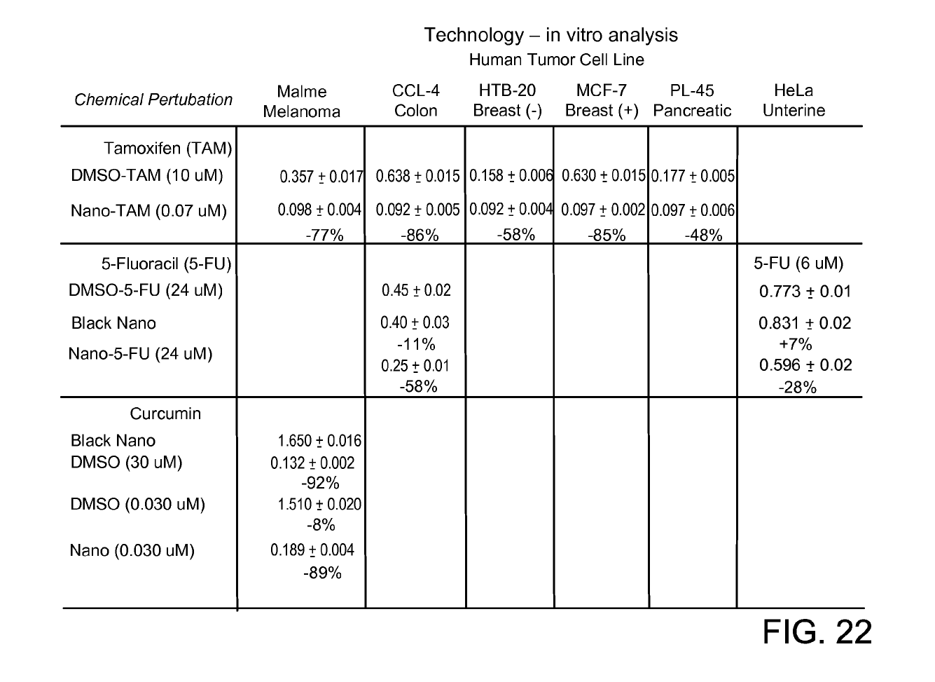

FIG. 11 shows the in vitro activity of both the curcumin nanoemulsion and the curcumin-DMSO mixture against melanoma cancer cells. The curcumin nanoemulsion was prepared by combining gelatin and EMG-20 or gelatin and Solutol® HS15 in a 1:1 weight ratio. The cell inhibition activity of the blank nanoemulsion, a 30 micromolar DMSO-curcumin mixture, a 0.030 micromolar DMSO-curcumin mixture, and a 0.03 micromolar curcumin nanoemulsion were contacted with melanoma cancer cell lines. Malme-3M cells are grown 60-80% confluency and exposed with curcumin formulations mentioned above and tested the cell confluency after 72 hrs using MTS Assay R. Referring to FIG. 11, 30 micromolar concentration of curcumin in DMSO inhibited melanoma cancer cell proliferation; however, if the concentration of curcumin in DMSO is reduced to 0.03 micromolar, efficacy against the melanoma cancer cells is lost. In contrast, the curcumin nanoemulsion retains inhibitory activity against the melanoma cancer cells at concentrations of 0.03 micromolar. These results are also summarized in the Table of FIG. 22. The curcumin encapsulated in the SANE system showed melanoma cell growth inhibition of up to 1000 times greater compared to the DMSO-Curcumin preparation at the same concentration.

### Example 4

### SANE Compositions Including 5-Fluorouracil (5-FU)

Nanoemulsions of 5-fluorouracil (5-FU) were prepared according to the method of Example 2, using the active component 5-FU instead of coumarin, a fish oil lipophilic component, a water hydrophilic component, and a surfactant including poly-oxyethylene esters of 12-hydroxystearic acid (e.g., Solutol® HS15). The active component 5-FU was dissolved in the lipophilic component: 0.065 g of 5-FU was weighed and mixed with 1 g of Fish oil. The mixture of 5-FU and fish oil was combined with 5 g (10 wt %) ethoxylated non-ionic surfactant, Solutol® HS15 (poly-oxyethylene esters of 12-hydroxystearic acid), and the mixture was heated, and stirred for 5 mins using a magnetic stirrer, until the two components visually formed a homogeneous mixture, and the temperature at this time was 50-60 degrees C. Distilled water was then added (total volume of the emulsion=50 ml) while the mixture was stirred, with continued heating. At this stage it formed an O/W emulsion. During heating, when the PIT (or HLB temperature) of the system was (65-70 degrees C., phase inversion zone), the surfactant was in equilibrium with the oil, and water phases. Heating and stirring was continued beyond the PIT up to 80° C. At this temperature the system inverted to a W/O emulsion. Once this stage was reached, the heating, and stirring was stopped. The emulsion was cooled at RT to obtain an O/W emulsion. The emulsion was cooled at 25-30° C. below the PIT to obtain kinetically stable O/W emulsions.

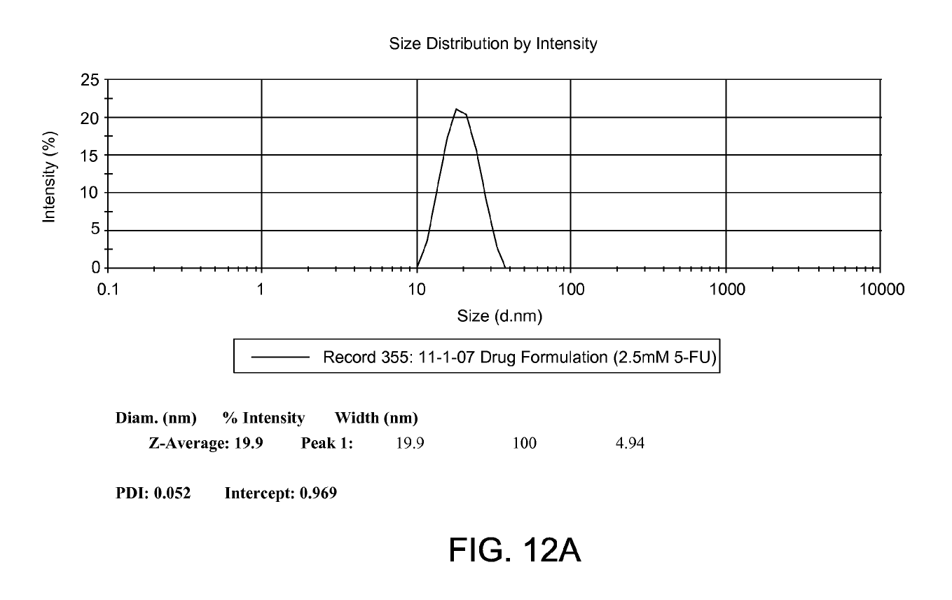

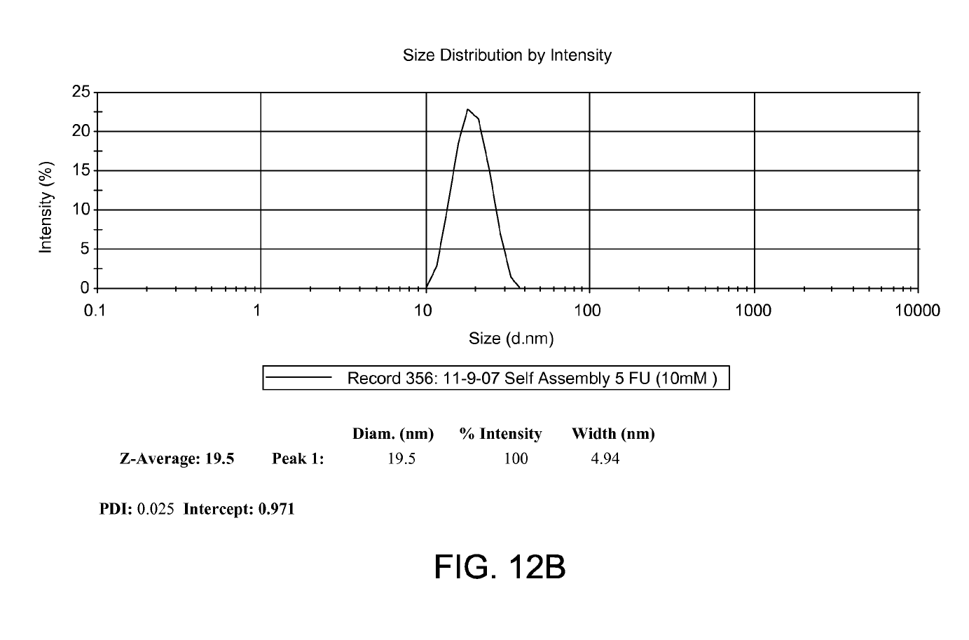

Two concentrations of 5-FU nanoemulsions including 5-FU/fish oil in the lipophilic component, Solutol® HS15 surfactant and water were prepared with average particle sizes of about 19 nm, measured by dynamic laser light scattering particle size analysis. FIG. 12A shows the particle size distribution of a 2.5 mM 5-FU SANE composition with an average droplet size of 19.9 nm and a size distribution peak width of 4.94 nm and a PDI of 0.052; FIG. 12B shows the particle size distribution of a 10 nM 5-FU SANE composition with an average droplet size of 19.5 nm and a size distribution peak width of 4.94 nm and a PDI of 0.025. The 5-FU nanoemulsions appeared transparent and remained steady even 2 months later at the room temperature. Particle size distributions measured for the SANE formulation of 5 FU at 2.5 mM and 10 mM concentrations, respectively illustrated virtually identical particle size and homogeneity despite the 4 fold difference in 5 FU concentration. TEM images of the 5 FU SANE composition showed small particles of relatively uniform and homogenous morphologies compared to large and irregular particle sizes observed in a mixture of 5FU with DMSO

The in vitro activity of the 5-FU nanoemulsion was measured against three cancer cell lines (CCL-221, Malme-3M, and CCL-2) using a Cell Proliferation Assay (MTS Assay). The MTS Assay uses a tetrazolium compound (MTS) and an electron coupling reagent (PES) was purchased from Fisher Scientific (Pittsburgh, Pa.). The tetrazolium compound (MTS) can be bioreduced into a colored formazan product by dehydrogenase enzymes in metabolic cells. Formazan is a water-soluble compound and can be dissolved in the culture medium, the quantity of formazan product as measured by the absorbance at 490 nm was directly proportional to the number of living cells in culture.

To perform the MTS Assays, approximately 4000 cells/well of each cancer cell line (CCL-221, Malme-3M, and CCL-2) were counted and placed in the 96 well plate with 100 microliter cell culture media. After incubation for 5 hours, the cells were attached to the bottom of the well and the diluted nanoemulsions were then added and incubated for 48 hours. Subsequently, the media were removed by aspiration and 100 microliter of fresh phenol free cell culture media was pipetted followed by adding 20 microliter of MTS reagent into each well. The plate was incubated for 2 hours at 37 degrees C. in a humidified, 5% CO2 atmosphere and the 96-well plate reader was used to measure the absorbance at 490 nm. The 5-FU nanoemulsions prepared in this study had 20 nm average particle size.

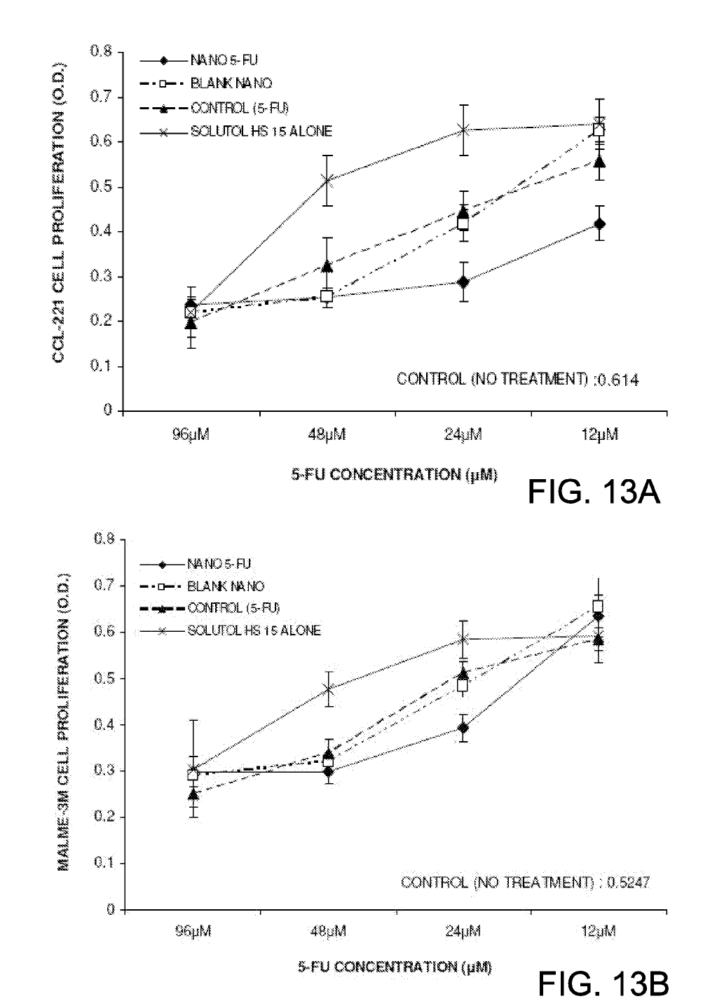

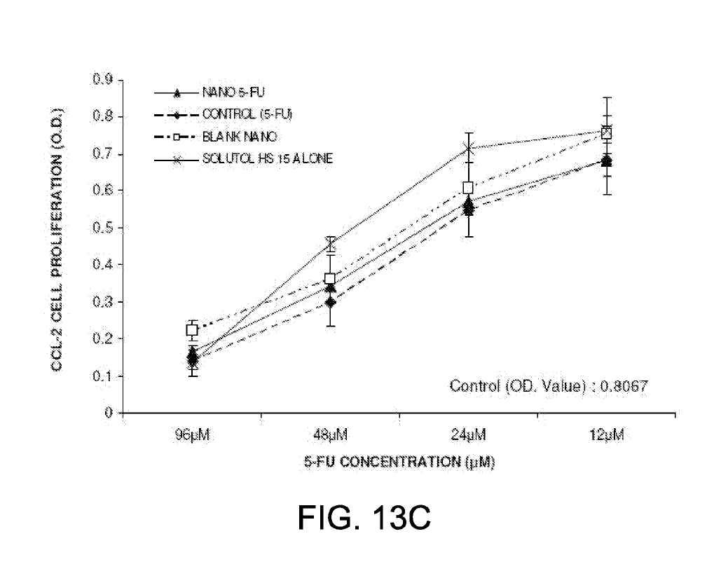

FIG. 13A is a graph showing the effect of 5-FU nanoemulsion on cell proliferation of colon cancer cell lines (CCL-221). Compared to non-nanoemulsion 5-FU, 5-FU nanoemulsion prevented cell proliferation of CCL-221 at 24 μM (−35%, p<0.001) and 12 μM (−25%, p=0.011). For all groups, N=9 and error bar was represented by SEM. FIG. 13B is a graph showing the effect of 5-FU nanoemulsion on cell proliferation of melanoma cancer cell lines (Melma-3m). Compared to non-nanoemulsion 5-FU, 5-FU nanoemulsion prevented cell proliferation of Melma-3m up to 24% at 24 μM(p<0.001). For all groups, N=9 and error bar was represented by SEM. FIG. 13C is a graph showing the effect of 5-FU nanoemulsion on cell proliferation of cervical cancer cell lines CCL-2). For all groups, N=9 and error bar was represented by SEM.

The melanoma cancer cells (Malme-3M) and colon cancer cells (CCL-221) were treated with appropriate dilutions of 10 mM 5-FU nanoemulsion and the efficacy of 5-FU nanoemulsion was compared to the non-encapsulation 5-FU at 96 μM, 48 μM, 24 μM, and 12 micromolar. 5-FU nanoemulsion was found to possesses better efficacy in preventing cell proliferation of CCL-221 at 24 μM (−35%, p<0.001) and 12 μM (−25%, p=0.011) as compared to non-encapsulation 5-FU (FIG. 13A). Additionally, Malme-3M cell proliferation was effectively inhibited by 5-FU nanoemulsion up to 24% at 24 μM (p<0.001) (FIG. 13B) whereas non-encapsulation 5-FU (i.e., 5-FU that was not formulated in a nanoemulsion) was not as effective at the same concentration. As to cervical (CCL-2) cancer cells, 5-FU nanoemulsion performed the same trend as non-encapsulation 5-FU (FIG. 13C).

The cell culture experiments demonstrated that 5-FU nanoemulsion provide increased efficacy against melanoma cancer cells (Malme-3M) and colon cancer cells (CCL-221) at lower concentrations than 5FU that is not incorporated in a nanoemulsion. The results indicate that the 5-FU nanoemulsion can provide a 5-FU delivery vehicle that enhances the efficacy of 5-FU against both Malme-3M and CCL-221 cancer cell lines. This suggests that the dosage of 5-FU effective in the conventional administration without a nanoemulsion could be reduced if administered as a nanoemulsion formulation. This is especially beneficial to the patients since using lower level of chemotherapeutic agents are generally safer and are associated with few adverse sides effects. Although nanoemulsions were found to effectively enhance 5-FU in both Malme-3M and CCL-221 cancer cell lines, less or no enhanced effects were observed when 5 FU was delivered as a nanoemulsion to CCL-2 cancer cells. This can be due to the drug specificity against certain cancer cell lines. The physicochemical properties of emulsions (e.g., stability and release characterization) can be highly dependent on the size of emulsified droplets that they contain.

FIG. 22 is a table summarizing in vitro effects of SANE compositions including tamoxifen, 5-FU, and Curcumin on Malme Melanoma Cells, CCL-4 Colon Cancer Cells, HTB-20 Cells, MCF-7 Cells, PL-45 Pancreatic Cells, and/or HeLa Uterine Cells.

This study indicates that SANE compositions including the active component 5-FU have higher efficacy at lower doses against melanoma cancer cells (Malme-3M) and colon cancer cells (CCL-221) than 5-FU that is not formulated as a nanoemulsion. These results indicate that 5-FU SANE compositions can be formulated with lower therapeutically effective doses than are generally required in 5-FU formulations that are not SANE compositions. Accordingly, SANE compositions comprising chemotherapeutic drugs at lower therapeutically effective doses can be safer and be associated with few adverse sides effects that formulations of the same chemotherapeutic drugs outside of a SANE composition.

### Example 5

### SANE Compositions Including Dacarbazine

This example describes the formation of SANE compositions that encapsulate the lipid soluble anti-melanoma drug dacarbazine with particle sizes of up to 20 nm. Nanoemulsions of the cytotoxic antineoplastic cancer drug dacarbazine (i.e., 5-(3,3-Dimethyl-1-triazenyl)imidazole-4-carboxamide, CAS 4342-03-4) were prepared by mixing 0.091 g of DAC (10 mM) with 1 g of Fish oil, respectively, then adding 5 g (10 wt %) of the ethoxylated non-ionic surfactant Solutol® HS15 (poly-oxyethylene esters of 12-hydroxystearic acid) with low toxicity in vivo. The resulting mixture was heated and stirred for 5 mins using a magnetic stirrer, until the two components visually form a homogeneous mixture and the temperature at this time should be at 50-60 degrees C.

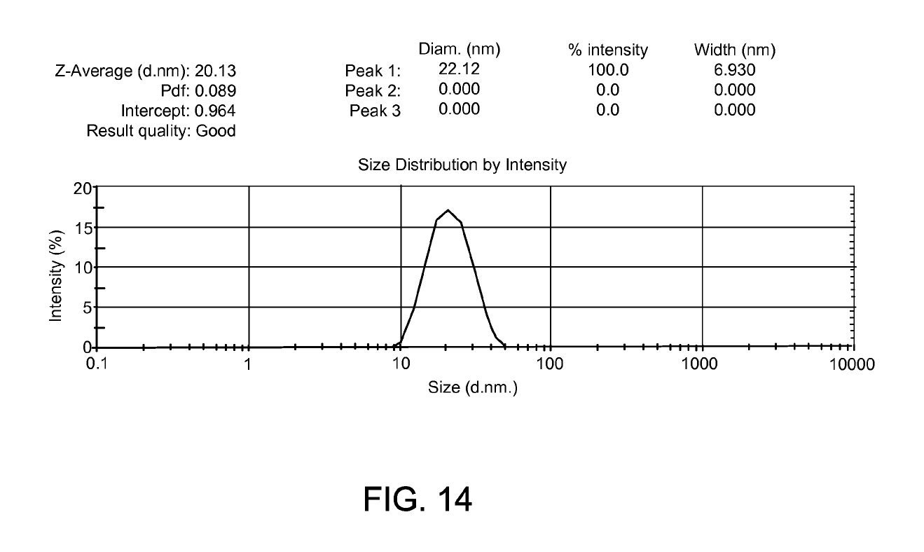

Distilled water was then added (total volume of the emulsion=50 ml) to the mixture while heating and stirring the mixture. At this stage, the mixture formed an O/W emulsion. During heating, when the PIT (or HLB temperature) of the system was reached (65-70 degrees C., phase inversion zone), the surfactant was in equilibrium with the oil and water phases. Heating and stirring was continued beyond the PIT up to 80° C. At this temperature, the system inverted to a W/O emulsion. Once this stage was reached the heating and stirring was stopped. The emulsion was cooled at RT to obtain an O/W nanoemulsion. The nanoemulsion should be cooled at 25-30° C. below the PIT to obtain kinetically stable O/W dacarbazine nanoemulsions. The resulting dacarbazine nanoemulsions had an average fish oil droplet size of 20.13 nm, and a droplet size distribution width of about 6.93 nm and a PDI of 0.089 (FIG. 14). When this procedure was repeated using comparable amounts of rice bran oil instead of fish oil, a stable nanoemulsion did not form.

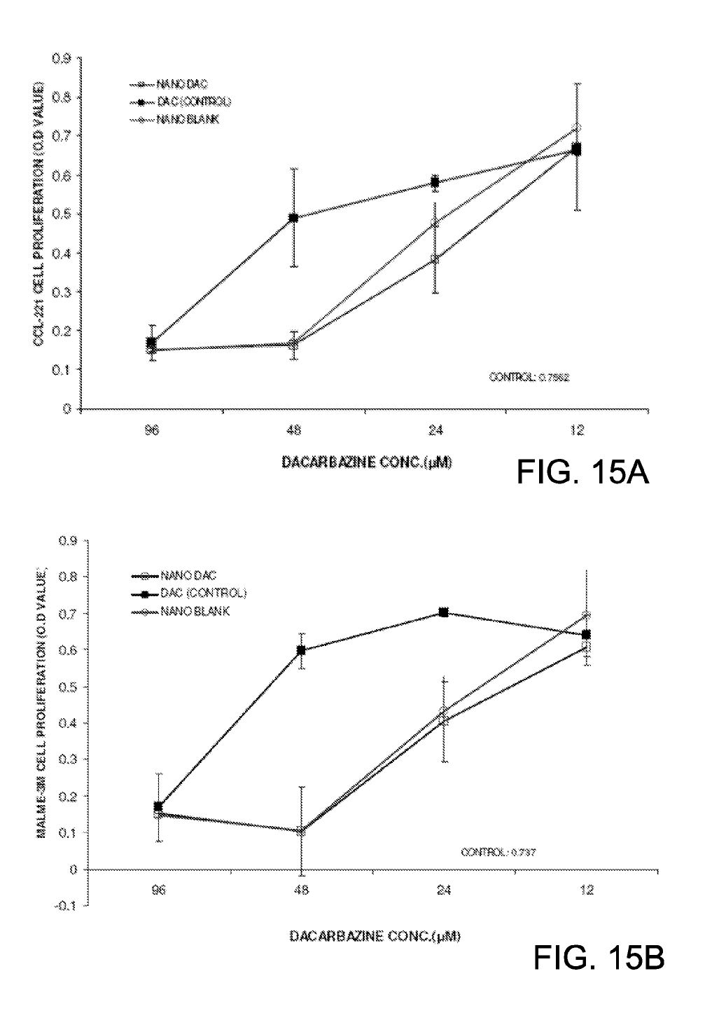

The in vitro activity of the dacarbazine-fishoil-Solutol® HS15 nanoemulsion was tested against melanoma cancer cells (Malme-3M) and colon cancer cells (CCL-221) treated with appropriate dilutions of 10 mM DAC nanoemulsion and the efficacy of DAC nanoemulsion was compared to the 5-FU (not formulated as a nanoemulsion) at 96 μM, 48 μM, 24 μM, and 12 μM. For comparison, the activity of mixtures of 5% dacarbazine in DMSO were also tested as controls. “Nanoblank” data was obtained from a mixture of 1 g fish oil, 5 g Solutol® HS15, and water up to 50 mL. Results are shown in FIG. 15A (the effect of DTIC nanoemulsion on cell proliferation of colon cancer cell Line CCL-221) and FIG. 15B (the effect of DTIC nanoemulsion on cell proliferation of skin cancer cell line Malme-3M). The DAC nanoemulsion was found to possesses improved efficacy in preventing cell proliferation of CCL-221 at 48 μM.

### Example 6

### SANE Compositions Including Paclitaxel

A pharmaceutically acceptable nanoemulsion formulation including paclitaxel with enhanced water solubility, bioavailability and efficacy was prepared. The nanoemulsion formulation of paclitaxel was prepared according to the method described with respect to FIG. 1 above, except that paclitaxel was dissolved in the lipophilic component before combination with the surfactant. The resulting paclitaxel nanoemulsions had an average particle size of about 20 nm. These formulations can be useful, for example, as a mitotic inhibitor used in cancer chemotherapy (e.g., to treat patients with lung, ovarian, breast cancer, head, and neck cancer).

We formulated paclitaxel in a self-assembled nanoemulsion (SANE) of oil and surfactant which was further used to study the effects of self assembled nanoemulsion on proliferation rates of colon and pancreatic cancer. Paclitaxel was purchased from Sigma (Cat number:417017). Rice Bran oil and Solutol® HS15 were purchased from Select origins and BASF respectively. The cell lines used were purchased from ATCC. All other reagents were analytical grade or higher and obtained from commercial sources.

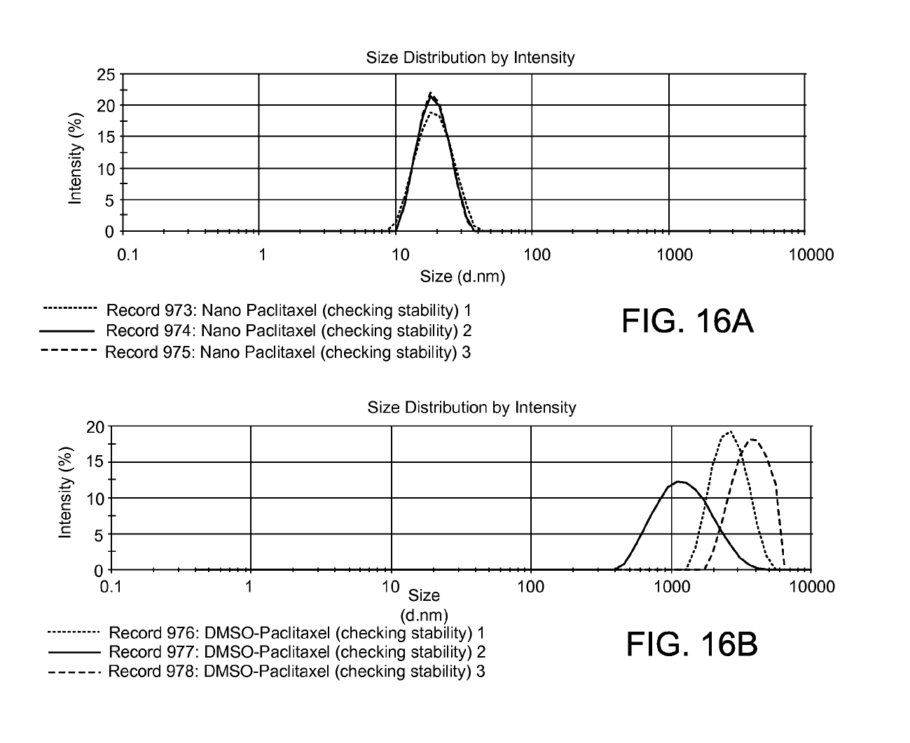

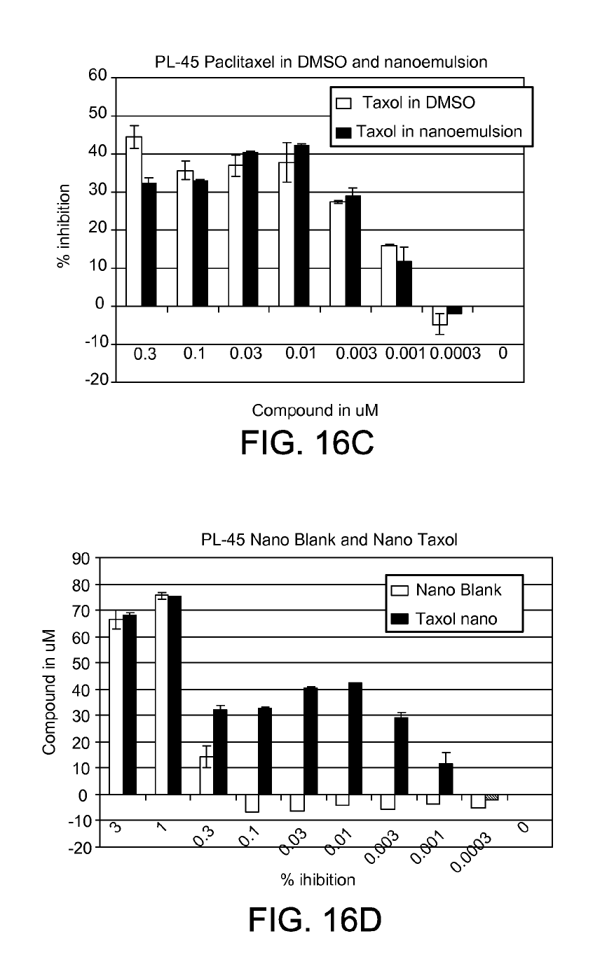

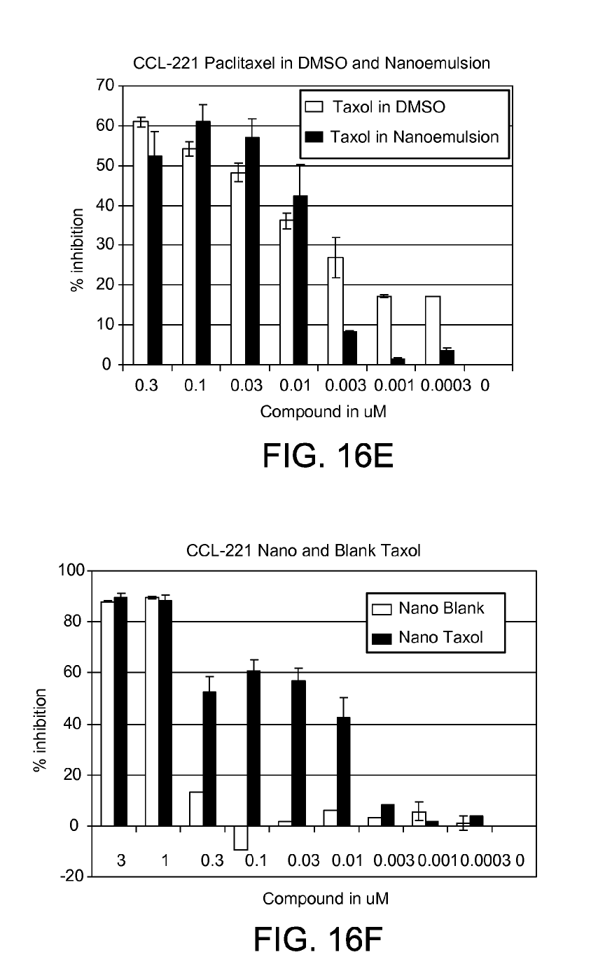

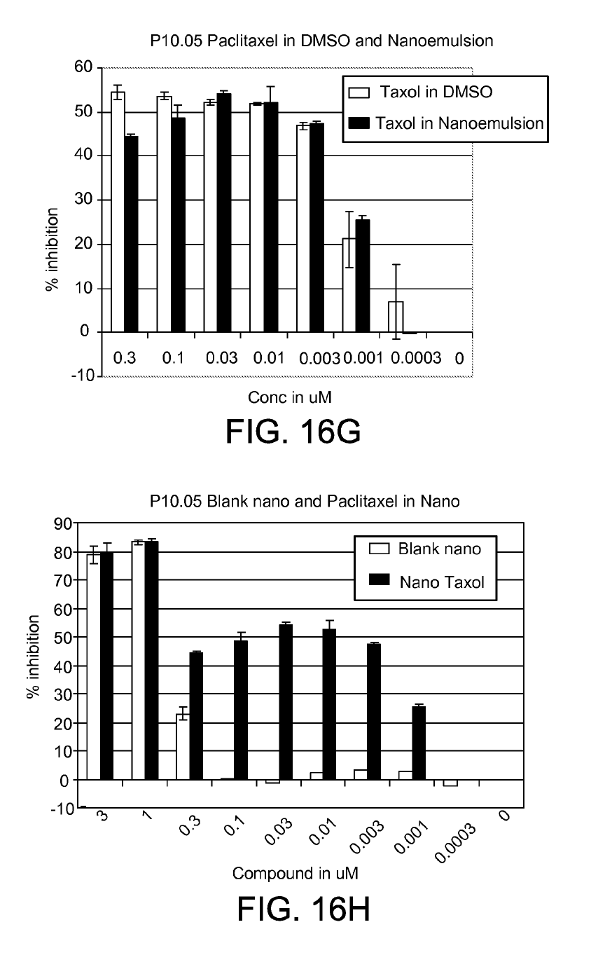

A specified amount of the oil was weighed in a vessel. The mixture was heated and stirred for 5 minutes using a magnetic stirrer, until the paclitaxel visually appeared to have dissolved in oil at about 50-60 degrees C. A specific amount of ethoxylated non-ionic surfactant is added. The PIT (also referred to as HLB temperature) was identified based on the HLB number (Hydrophile-Lipophile balance) of the surfactant. PIT increases with increase in HLB number. The mixture was heated and stirred again for 5 minutes at about 50-60 degrees C. until the three components form a homogeneous mixture. Distilled water (total volume=50 ml) was added while stirring the mixture at about 60 degrees C. At this stage the mixture formed an O/W emulsion. During heating when the PIT (or HLB temperature) of the system is reached (65-70 degrees C., phase inversion zone), the surfactant is in equilibrium with the oil and water phases. Heating and stirring was continued beyond the PIT up to 80° C. At this temperature, the system inverts to a W/O emulsion. The emulsion is cooled to room temperature obtain an O/W nanoemulsion including paclitaxel. FIG. 16A shows the particle size distribution of three separate paclitaxel nanoemulsions, each having an average oil droplet size of about 20 nm. FIG. 16B shows the particle size distribution for three mixtures of paclitaxel with DMSO (not nanoemulsions), having average particle sizes of about 1 micrometer (1,000 nm) or greater.

The in vitro activities of the paclitaxel nanoemulsions were tested against four human cancer cell lines PL-45 (FIG. 16C), CCL-221 (FIG. 16E), and P10.05 (FIG. 16G) indicated (a) that this formulation was as effective as dimethyl sulfoxide (DMSO) in inhibiting cell proliferation in various cancer cell lines and (b) nanoemulsion formulations of paclitaxel typically used for chemo-therapy treatment for breast cancer demonstrated very striking inhibition of cell proliferation in a pancreatic cancer cell line. For comparison, the inhibitory activity of comparable blank nanoemulsions (i.e., the same SANE compositions without any active component) and a mixture of DMSO and paclitaxel were tested for inhibition of the PL-45 (FIG. 16D), CCL-221 (FIG. 16F), and P10.05 (FIG. 16H) cell lines. The paclitaxel SANE compositions exhibited higher inhibitory effect than the mixture of paclitaxel in DMSO for 0.01-0.03 micromolar concentrations against all three cell lines. Furthermore, SANE compositions with paclitaxel doses of 0.003 micromolar were more effective against PL-45 and P10.05 cells than the same dose of paclitaxel in DMSO (FIGS. 16C, 16E, and 16G).

FIG. 16C is a graph showing dose-dependant anti cancer activity of paclitaxel in DMSO and paclitaxel in a nanoemulsion made up of rice bran oil and Solutol® HS15 against PL-45 cells after 48 hours of treatment. The graphs indicate a 44% and 40% growth inhibition when treated with paclitaxel in DMSO and paclitaxel in nanoemulsion respectively. Compared to the suspension of paclitaxel, the nanoemulsion of paclitaxel had comparable effects on the cell proliferation. The error bars represent the standard error of mean for experiments done in duplicates. Thus this indicates (a) this formulation was as effective as dimethyl sulfoxide (DMSO) a solubilizing agent (which cannot be used in humans because of its toxicity), and (b) nanoemulsion formulations of paclitaxel typically used for chemo-therapy treatment for breast cancer and ovarian cancer demonstrated very striking inhibition of cell proliferation in a pancreatic cancer cell line. FIG. 16D compares empty nanoemulsion control to the nanoemulsion preparations of paclitaxel indicating that the cell inhibition activity of the 0.3 uM paclitaxel comes from the drug in the nanoemulsion and not the nanoemulsion itself which indicates the increased growth inhibition of the cells is due to the activity of paclitaxel encapsulated in nanoemulsion and not the empty nanoemulsion.

FIG. 16E demonstrates dose-dependant anti cancer activity of paclitaxel in dimethyl sulfoxide (DMSO) and paclitaxel in nanoemulsion made up of rice bran oil and Solutol® HS15 against CCl-221 cells after 48 hours of treatment. The graphs indicate a 60% and 52% growth inhibition when treated with paclitaxel in DMSO and paclitaxel in nanoemulsion respectively. Compared to the suspension of paclitaxel nano-paclitaxel had comparable effects on the cell proliferation. The error bars represent the standard error of mean for experiments done in duplicates. Thus, this indicates (a) this formulation was as effective as DMSO, a solubilizing agent which cannot be used in humans because of its toxicity, and (b) nanoemulsion formulations of paclitaxel typically used for chemo-therapy treatment for breast cancer and ovarian cancer demonstrated very striking inhibition of cell proliferation in a colon cancer cell line. FIG. 16F compares empty nanoemulsion control to the nanoemulsion preparations of paclitaxel indicating that the cell inhibition activity of the 0.3 uM paclitaxel comes from the drug in the nanoemulsion and not the nanoemulsion itself which indicates the increased growth inhibition of the cells is due to the activity of paclitaxel encapsulated in nanoemulsion and not the empty nanoemulsion.

FIG. 16G demonstrates dose-dependant anti cancer activity of paclitaxel in DMSO and paclitaxel in nanoemulsion made up of rice bran oil and Solutol® HS15 against P10.05 cells after 48 hours of treatment. The graphs indicate a 60% and 52% growth inhibition when treated with paclitaxel in DMSO and paclitaxel in nanoemulsion respectively. Compared to the suspension of paclitaxel nano paclitaxel had comparable effects on the cell proliferation. The error bars represent the standard error of mean for experiments done in duplicates. Thus this indicates (a) this formulation was as effective as DMSO a solubilizing agent which cannot be used in humans because of its toxicity (b) nanoemulsion formulations of paclitaxel typically used for chemo-therapy treatment for breast cancer and ovarian cancer demonstrated very striking inhibition of cell proliferation in a pancreatic cancer cell line. FIG. 16H compares empty nanoemulsion control to the nanoemulsion preparations of paclitaxel indicating that the cell inhibition activity of the 0.3 uM paclitaxel comes from the drug in the nanoemulsion and not the nanoemulsion itself which indicates the increased growth inhibition of the cells is due to the activity of paclitaxel encapsulated in nanoemulsion and not the empty nanoemulsion.

In addition, lower dose paclitaxel SANE compositions having 0.001 micromolar paclitaxel showed greater inhibition of P10.05 cells compared to the same dose of paclitaxel in DMSO (FIG. 16G), while higher dose paclitaxel SANE compositions having 0.1 micromolar paclitaxel levels showed greater inhibition of CCL-221 cells compared to the same dose of paclitaxel in DMSO (FIG. 16E). In conclusion, using established cell lines from various cancers, a water-soluble nanoemulsion formulation of paclitaxel with a particle size of 20 nm was very effective in inhibiting cell proliferation.

### Example 7

### SANE Compositions Including Tocotrienols

Nanoemulsions including tocotrienols were prepared according to the method of Example 2 using rice bran oil as the lipophilic component and SolultolHS15 (Macrogol hydroxystearate) as the surfactant, except that tocotrienols were used as the active component instead of coumarin. Tocotrienols (Eastman Chemical Co.) were used in the form of a dense oil. Together with tocopherols form a component of vitamin E. Natural tocotrienols exist in four different forms or isomers, named alpha-, beta-, gamma-, and delta-tocotrienol, which contain different number of methyl groups on the chromanol ring. Briefly, the tocotrienols (5 mg) was dissolved in rice bran oil (1 g) while heating to form the lipophilic component, which was then combined with the Solutol® HS15 surfactant (5 g) to form a first composition. The first composition was diluted with water (44 ml) to form an O/W macroemulsion, which was heated above the PIT of the surfactant to form a W/O emulsion. The tocotrienol nanoemulsion was formed by rapidly cooling the W/O emulsion to room temperature. Other tocotrienol nanoemulsions also formed with surfactant to rice bran oil initial weight ratios of 1:1 to 5:1, with a S/O weight ratio of 5:1 providing a stable formulation. The tocotrienol nanoemulsion can be useful, for example, for treating high cholesterol. When this procedure was repeated using a comparable amount of the ethoxylated monoglyceride EMG-20 as a surfactant instead of Solutol® HS15, a stable nanoemulsion did not form. For comparison testing, a mixture of 50 mL DMSO and tocotrienols (5 mg) was also formulated.

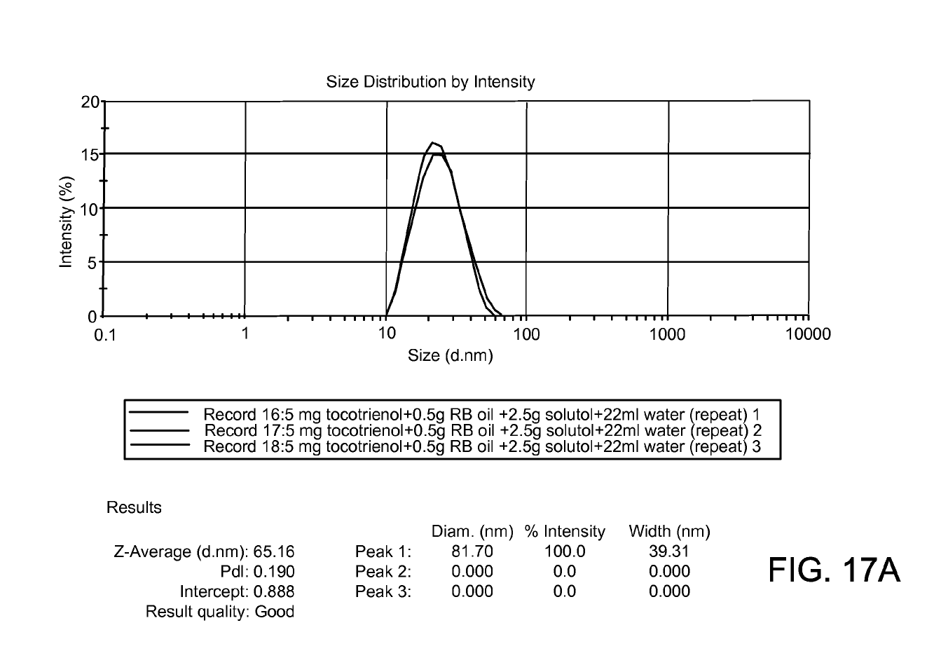

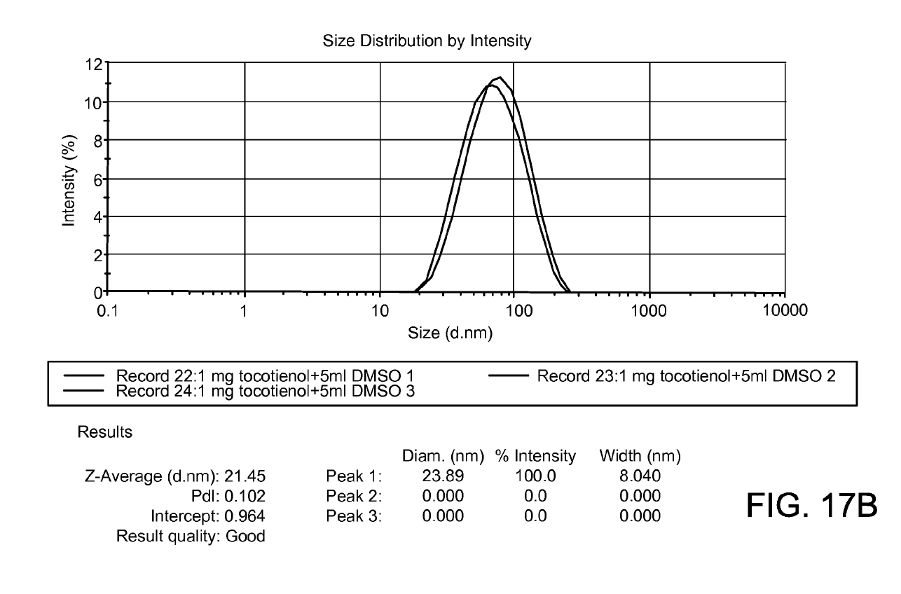

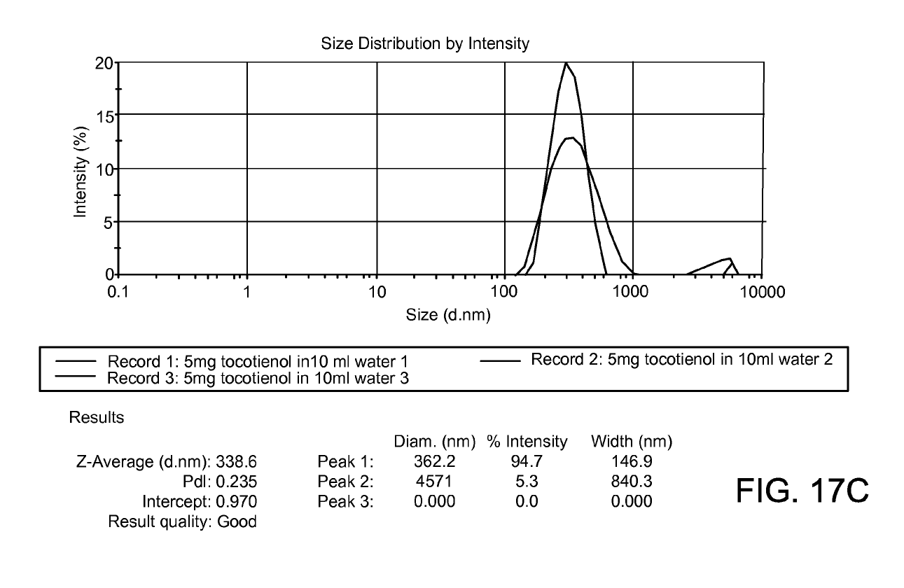

FIG. 17A is a graph of particle size distribution measured for three of the following SANE composition including tocotrienol: 5 mg tocotrienol, 0.5 g rice bran oil, 2.5 g Solutol® HS15 surfactant, and 22 mL deionized water. The Z-average particle size in the three tocotrienol SANE compositions was 65.16 nm with a peak width of 39.31 nm and a PDI of 0.190. FIG. 17B is a graph of particle size distribution for a mixture of 1 mg tocotrienols in mL DMSO. The Z-average particle size of these three mixtures was 23.89 nm with a peak width of 8.04 nm and a PDI of 0.102. FIG. 17C is a graph of particle size distribution for three mixtures of 5 mg tocotrienols in 10 mL water. The Z-average particle size of these three mixtures was 338.6 nm with a peak width of 146.9 nm and a PDI of 0.235. The tocotrienol SANE compositions measured to obtain data for FIG. 17A showed smaller average particle size than the DMSO-tocotrienol composition measured in FIG. 17B or the water-tocotrienol composition measured in FIG. 17C.

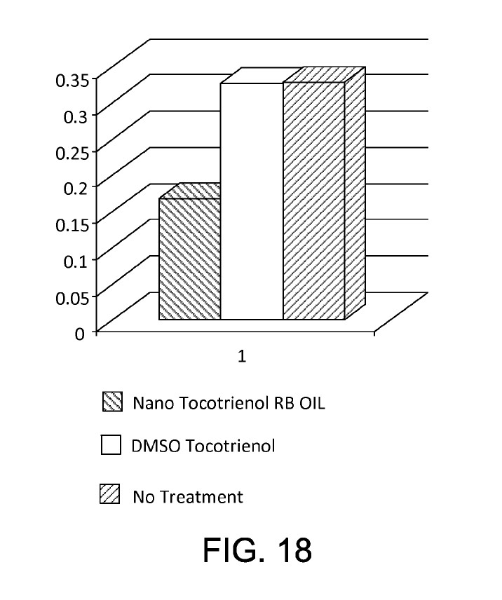

The effect of the tocotrienol nanoemulsion on inhibiting cholesterol in HepG cells was compared to the effect of the DMSO-tocotrienol mixture on the HepG cells. HepG cell line (obtained from Atcc.org) express HMGCoA reductase which is involved in cholesterol synthesis. The HepG cells were exposed with Formulations of DMSO Tocotrienols, NanoTocotrienols and No-Treatment (NT)(Added 10 ul of each formulation). Cells were not exposed to any growth factors or insulin. The cells were grown in the experimental conditions for 72 hrs (with formulations) and next 72 hours with fresh media with out formulations, and then lysed with RIPA buffer for cholesterol analysis. Data is shown in Table 3 below and FIG. 18, in relative absorption units from UV-VIS measurement. A 47% inhibition of cholesterol was observed when exposing the HepG cells to the tocotrienol compared to 0% with the DMSO-tocotrienol mixture. The reading of 0.33 UV-VIS absorption units in the table below corresponds to 88 mg cholesterol/di; a reading of 0.17 corresponds to 45 mg cholesterol/dL.

TABLE 3
Cholesterol Levels Measured After Treatment
(UV-VIS Relative Absorption Units)

| Nano | | |
|---|---|---|
| Tocotrienol | DMSO | |
| RBoil | Tocotrienol | No Treatment |
| 0.17 | 0.33 | 0.33 |
| 0.17 | 0.32 | 0.32 |
| 0.16 | 0.32 | 0.33 |
| 0.17 | 0.33 | 0.33 |

% inhibition of NanoTocotrienol in RB OIL 47%
% inhibition of DMSO Tocotrienol 0

### Example 8

### SANE Compositions Including Lutein

Nanoemulsions including a lutein ester (Xangold; Cognis, Cincinnati, Ohio) were prepared according to the method of Example 2 using soybean oil as the lipophilic component and C20 ethoxylated monoglyceride EMG-20 as the surfactant, except that lutein ester was used as the active component instead of coumarin. Briefly, the lutein ester (7.57 mg) was dissolved in soybean oil (1 g) while heating to form the lipophilic component, which was then combined with the C20 ethoxylated monoglyceride (EMG-20) surfactant (5 g) to form a first composition. The first composition was diluted with water (44 ml) to form an O/W macroemulsion, which was heated above the PIT of the surfactant to form a W/O emulsion. The lutein nanoemulsion was formed by rapidly cooling the W/O emulsion to room temperature. Stable lutein nanoemulsions (e.g., after 2 days) formed by the procedure discussed with respect to FIG. 1 using formulations with the EMG20 surfactant to soybean oil at initial weight ratios of 5:1 and 6:1. When this procedure was repeated using a comparable amount of the rice bran oil instead of soybean oil as the lipophilic component, a stable nanoemulsion did not form from formulations having surfactant to oil ratios of 3:1, 4:1, 5:1 or 6:1 heated to a maximum temperature of about 70° C. This can be due to the fact that the soybean oil was higher in polyunsaturated fats (about 58% in the soybean oil, compared to about 39% in the rice bran oil) and/or the structure of lutein which is an oxygenated carotenoid having double bonds. Lutein nanoemulsions can comprise soybean oil and lutein as the lipophilic component, water or other aqueous hydrophilic component and a ethoxylated monoglyceride surfactant with about a 5:1 initial weight ratio between the surfactant and the oil.

### (Comparative) Example 9

### SANE Compositions Including siRNA

A series of siRNA nanoemulsions were prepared according to the method described with respect to FIG. 1 from a formulation of gelatin (1 gm) (animal gelatin, Sigma) and siRNA (siGlo, a fluorescent form of Lamim A/C) in the lipophilic component, an ethoxylated monoglyceride (EMG-20) surfactant (1 gm), and 48 mL distilled/deionized water. The siRNA was obtained from a company called Dharmacon 2650 Crescent Drive, #100 Lafayette, Colo. (e.g., Thermo Fisher Scientific Catalog Number D-001620-02-05; target accession number NM_{—}170708 (Human)). The siRNA used in this Example was obtained from Dharmacon was siGLO Lamin A/C Control siRNA (Human) Cat # D-001620-02-05. The nanoemulsion was made by weighing out 1 gm of gelatin melting it with constant stirring. Then 1 gm of EMG was added by constantly mixing the mixture on low heat 50 microliters of 20 micromolar control siRNA was added to the mixture with constant stirring. The total volume was made up to 50 milliliters by adding 48 milliliters of distilled water with constant mixing at low heat. The emulsion was stirred for 20 minutes.

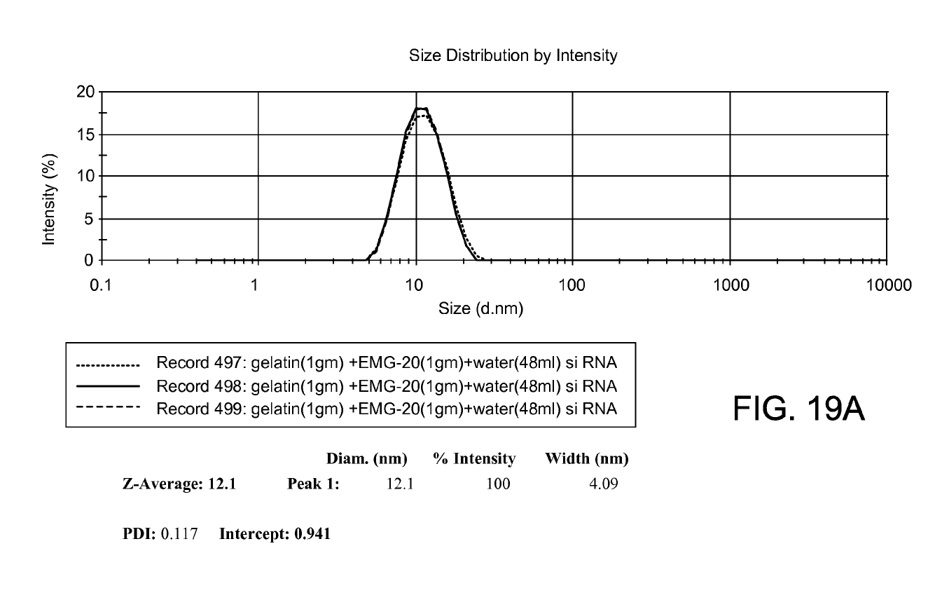

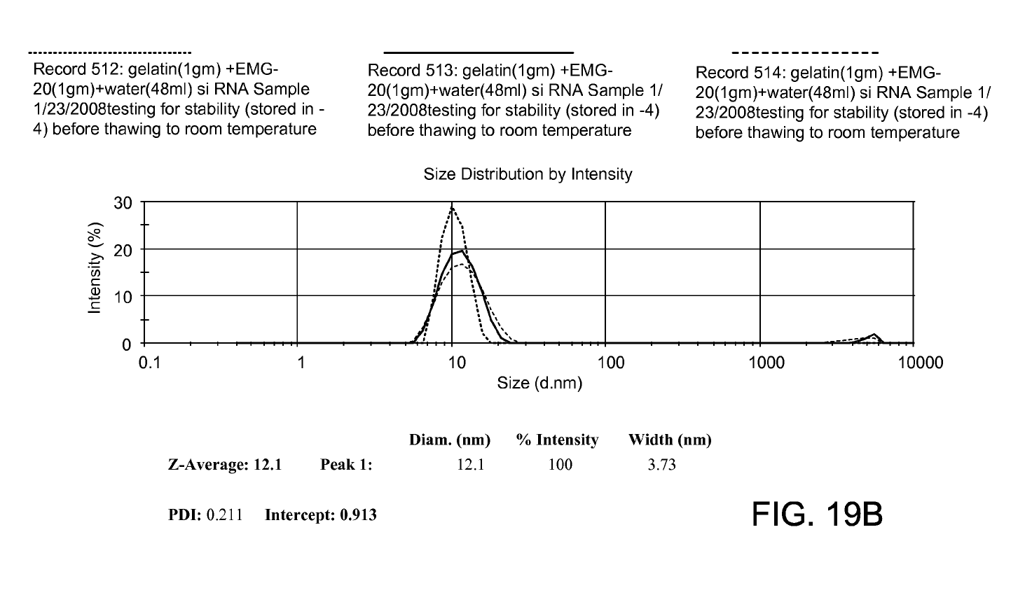

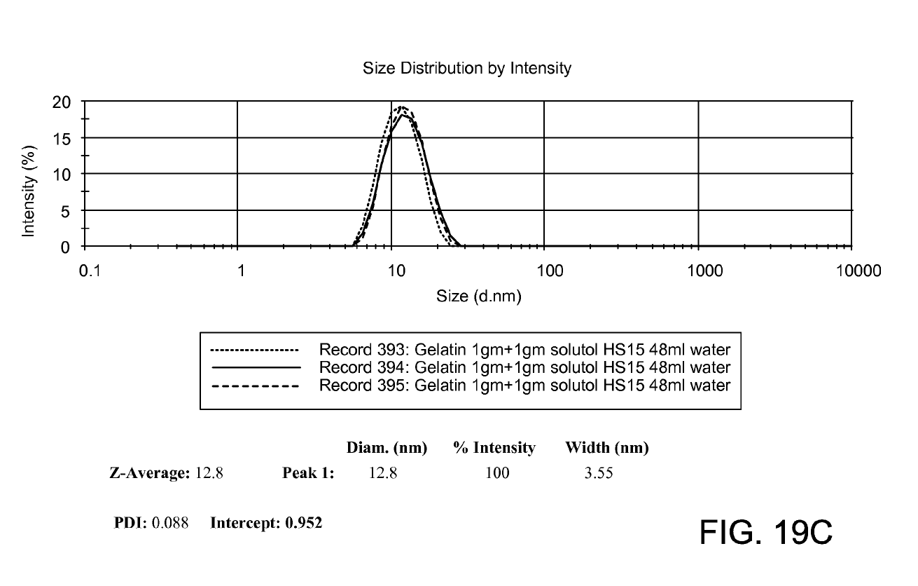

The particle size of a first siRNA/gelatin particles formed at room temperature in the resulting nanoemulsion had an average size of 12.1 nm, with a peak width of about 4.1 nm and a PDI of 0.117 (FIG. 19A). A series of three siRNA nanoemulsions were stored at a temperature of −4 or −20° C., then thawed and the particle size of each siRNA/gelatin in the thawed nanoemulsion was measured an average size of 12.1 nm, with a peak width of about 3.7 nm and a PDI of 0.211 (FIG. 19B). FIG. 19B shows the particle size distribution for three tested samples each containing 1 gram gelatin, 1 gram EMG-20 surfactant, and 48 mL water with siRNA. However, when stored at −4° C. (FIG. 19B) and −20° C. and thawed at room temperature over a 3 hr period or heated to 50° C., they remain stable.

Another series of siRNA nanoemulsions were prepared according to the method described with respect to FIG. 1 from a formulation of gelatin (1 gm) and siRNA in the lipophilic component, a polyoxyethylene ester of 12-hydroxysteric acid such as Solutol® HS15 surfactant (1 gm), and 48 mL distilled/deionized water. The particle size of a first siRNA/gelatin particles formed at room temperature in the resulting nanoemulsion had an average size of 12.8 nm, with a peak width of about 3.6 nm and a PDI of 0.088 (FIG. 19C). The siRNA SANE compositions using Solutol® HS15 were more stable than those using EMG-20 as surfactant.

However, the siRNA nanoemulsion did not result in the uptake of siRNA by HeLa cells. Briefly, 5 nmol siRNA was diluted in 250 microliter of PBS to make up a stock of 20 micromolar. A siRNA SANE preparation was used as described above, with 100 microliter of 20 micromolar siRNA was added on to 25 mL of nano formulation (gelatin based formulation). The following siRNA SANE composition transfection protocol was followed for Cell Plating (HeLa cells were used for the following experiment):

1. Trypsinize and count cells.

2. Dilute cells in antibiotic-free medium to a plating density of 5.0×10⁴ cells/mL for transfection with DharmaFECT 1.

3. Plate 100 microliter of cells into each well of a 96-well plate.

4. Incubate cells at 37° C. with 5% CO2 overnight.

The following protocol was followed for transfection (for 100 nM of siRNA) (Performing experiments in triplicate is recommended. All calculations are shown for triplicate samples in 96-well format. To account for loss during pipetting, all volumes are multiplied by 3.5):

1. Prepare a 2 micromolar siRNA solution in 1×siRNA Buffer or another appropriate RNase-free solution.

2. In separate tubes, dilute 2 micromolar siRNA (Tube 1) and DharmaFECT 1 (Tube 2) with serum-free medium. For example, prepare the following:

a. Tube 1—Add 17.5 microliter of 2 μM siRNA to 17.5 microliter serum-free medium. The total volume is 35 microliter.

b. Tube 2—Add 1.4 microliter of DharmaFECT 1 to 33.6 microliter serum-free medium. The total volume is 35 microliter.

3. Mix the contents of each tube gently by pipetting carefully up and down and incubate for 5 minutes at room temperature.

4. Add the contents of Tube 1 to Tube 2. In this example, the total volume is 70 microliter. Mix by pipetting carefully up and down and incubate for 20 minutes at room temperature.

5. Add sufficient antibiotic-free complete medium to the mix in step 4 for the desired transfection volume. In this example, add 280 microliter for a total volume of 350 microliter.

6. Remove culture medium from the wells of the 96-well plate and add 100 microliter of the appropriate transfection mix to each well.

7. Incubate cells at 37° C. in 5% CO2 for 24-48 hrs (for mRNA analysis) or 48-96 hrs (for protein analysis).

8. If cell toxicity is observed after 24 hours, replace the transfection medium with complete medium and continue incubation. Cell viability can be determined with alamarBlue®, MTT, or other assays for metabolic activity.

9. The cells were then observed under the fluorescence microscope.

The cell plates were observed under a Zeiss fluorescent microscope. Specially modified, fluorescent RNA duplexes provide a reliable visual assessment of transfection success. Fluorescent signal was localized to the nucleus of the HeLa cells as an unmistakable marker for uptake efficiency.

Initial data revealed that the fluorescence signal localized to the nuclei of the cells and was detected in the control utilizing a concentration of 100 nM siRNA. In contrast, the same intensity of the fluorescence signal incorporated into the HeLa cells for the siRNA encapsulated in the nanoemulsion was achieved at a concentration of 1 nM suggesting a 100 fold increase in cellular uptake for the nanoemulsion delivered siRNA compared to the non-nanoemulsion control.

In subsequent tests, fluorescence signal localized to nucleus was detected in the control as well as the plates containing siRNA encapsulated in the nanoemulsion. The starting amount of siRNA was 20 micromolar in 50 milliliters. This would give a starting concentration of 0.4 micromolar/mL. The samples were then diluted 200 and 400 fold. So the final concentration used on the cells was 1 nM and 2 nM whereas the starting concentration of the control was 100 nM. Fluorescence detection in this case is a phenotypic endpoint. The result can also be confirmed by western blot. The siRNA used in this experiment can knockdown the expression of LaminA/C protein. The western blots can be set up using the LaminAlC antibody.

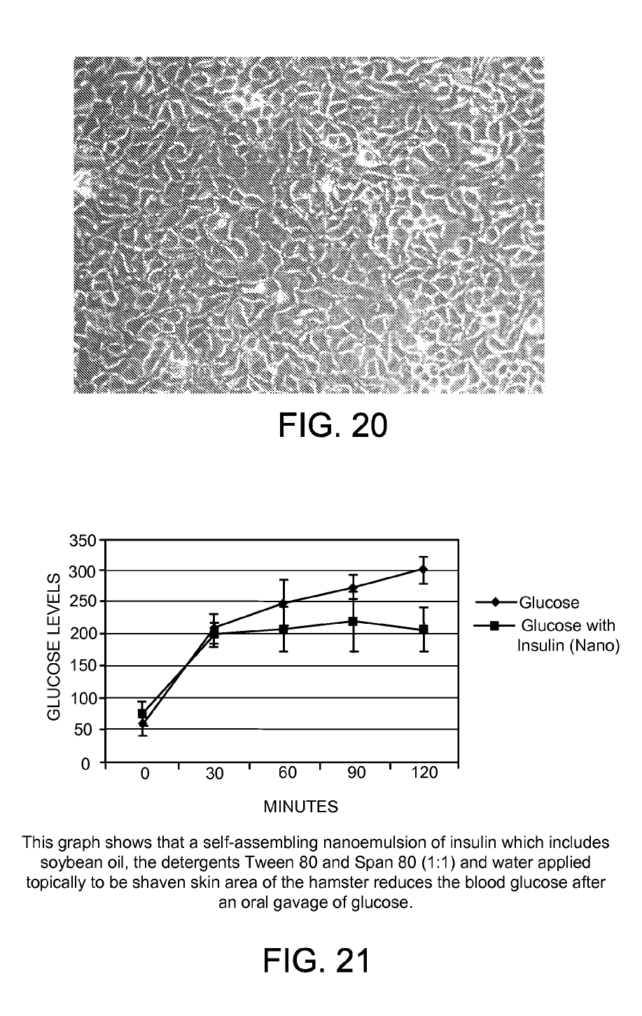

FIG. 20 is a fluorescence image of transfected HeLa cells after contact with the siRNA SANE composition, showing that a substantial amount of the siRNA composition was not taken into the HeLa cells. The uptake efficiency was determined by the uptake of fluorescent signal in the cell nucleus. If the siRNA uptake was effective, then the fluorescence tag would be seen in the nucleus. However, although the siRNA formed a nanoemulsion, FIG. 20 shows that the fluorescence was observed along the cell membrane indicating that the siRNA in the nanoemulsion formulation was not taken up by the cell.

### Example 10

### SANE Compositions Including Insulin

This example describes the formations of nanoemulsions with encapsulated proteins such a insulin and albumin with particle sizes of up to about 20 nm. Insulin can be delivered transdermal) with efficacy that prevents the rise in blood glucose following oral gavage. Water soluble proteins such as insulin were encapsulated in a SANE composition with particle sizes approximately 20 nm diameter. A SANE composition was prepared using a combination of Polysorbate 80 (also known TWEEN® 80) and Sorbitan monooleate (Span 80) (1:1), soybean oil, and water, and delivered transdermally into the blood stream of hamsters to reduce blood glucose levels after an oral gavage of glucose. Insulin can be delivered transdermally into the blood stream of hamsters to reduce blood glucose levels after an oral gavage of glucose. This Insulin SANE system is also able to deliver transdermally the water-soluble hormone insulin with sufficient efficacy to prevent the rise in blood glucose following the gavage of a glucose load in hamsters (FIG. 21). FIG. 21 is a graph illustrating blood glucose levels in hamsters after a transdermal SANE delivery of the water soluble protein hormone insulin following a glucose gavage. Notably, the lower blood glucose levels were observed after the transdermal delivery of the SANE-containing insulin immediately after the glucose gavage.

### Example 11

### SANE Compositions Including Lovastatin

This example describes formation of SANE compositions containing a lipid soluble active agent, to form a nanoemulsion that dramatically increases the efficacy in an in vitro cell culture system. Accordingly, a lipid soluble active agent can be formulated as a stable water dispersion. The resulting bioactive SANE composition can have increased efficacy compared to the active agent alone, allowing the preparation of therapeutically effective SANE compositions containing the active agent in a lower concentration than required to achieve a therapeutically effective dose of the active agent outside of the SANE composition. As a result, the SANE composition can be used to deliver the active agent with reduced adverse side effects associated with the active agent outside the SANE composition.

In particular, this example describes formation of a SANE composition containing the lipid soluble cholesterol-lowering HMG-CoA Reductase Inhibitor (Statin) as an active agent. The static containing SANE composition was observed to reduce cholesterol accumulation and HMG CoA Reductase activity in vitro in HEP G2 cells.

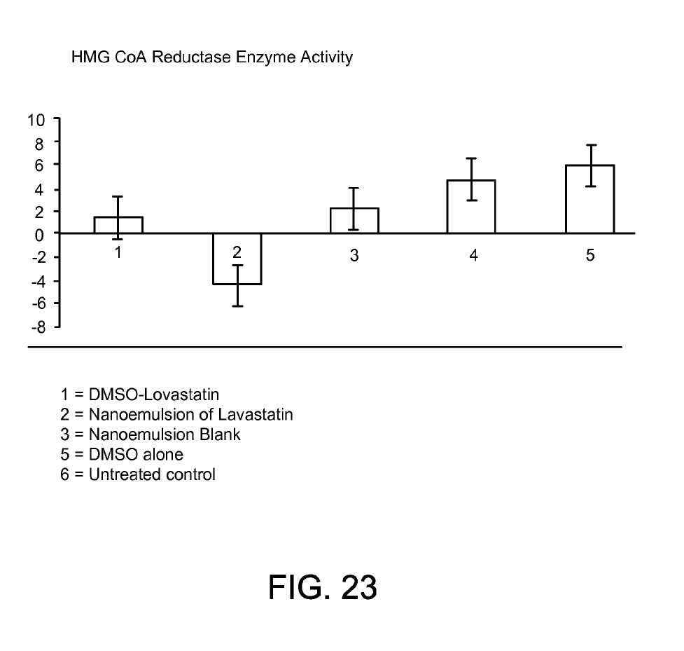

Results of these tests are shown in Tables 3 and 4 below and in FIG. 23. Each SANE composition was made by phase inversion temperature (PIT) method of a composition containing Rice Bran oil (Select Origins, Japan) as the lipophilic component and Solutol® HSI5 (BASF, USA as the surfactant with 44 mL of deionized water (Milli Q, Bedford, Mass., USA) as the hydrophilic component. Lovastatin was dissolved directly in dimethyl sulfoxide (DMSO, Sigma (St. Louis, M USA) to form a Nano-blank formulation for comparison, without the drug. Mean droplet size, wid and poly-dispersity index, were measured by Malvern Nano-S instrument (Malvern Instruments In Southborough, Mass., USA).

To obtain the data in Tables 4 and 5, and FIG. 23, the following compositions were made (a) a blank nanoemulsion with rice bran oil (0.5 gm), Solutol® HS15 surfactant (2.5 gm), and water (22 ml) formed by the SANE process in Example 1; (b) a lovastatin nanoemulsion made by adding 5 mg lovastatin to the composition used to make the blank nanoemulsion in composition (a made as a SANE composition according to Example 2, substituting 5 mg of lovastatin as the active component; and (c) a solution of lovastatin in DMSO (5 mg Lovastatin in 5 ml DMSO). A cell culture experiment with Hep 02 cells was performed using 10 microliters each of compositions (a) (b) 5 microliters of composition (c) to make equal concentration of the drug (450 nM), and a no treatment (NT) control experiments was performed.

Referring again to data shown in Tables 4 and 5 below, and in FIG. 23, a Cholesterol Assay, and HMG CoA reductase Assay was performed as follows: Hep 02 cells (ATCC, Manassas Va.) were plated in 70 mm plates and grown to 60-70% confluency .MEM (minimal essential medi (ATCC, Manassas, Va.>>, IO % FBS(Oemini bio products, USA), 5% CO₂ and at 37° C. were used to maintain cell growth conditions. To 10 microliters of the formulation was added 2 ul of insulin (Sigma (St. Louis, Mo., USA). After 72 hrs of incubation, cell lysates were made and stored at −80° The amount of cholesterol in the cells was measured using the Cholesterol E CHOD-DOAS method purchased from Wako Chemicals USA. HMO CoA Reductase activity was measured using the assay kit obtained from Sigma-Aldrich (St. Louis, Mo., USA). To perform Bradford and MTT assays, measurements were performed to (a) confirm that the starting number of cells were similar for all t different experimental conditions and (b) to show that the formulations were non-toxic to the cells respectively. The Bradford and MTT assay kits were purchased from Sigma Aldrich (St. Louis, Mo., USA).

TABLE 4

| Cholesterol Accumulation | | |
|---|---|---|
| in Hep-G2 Cells | | (% Reduction) |
| Control (No treatment) | 4.88 ± 0.24 mg/ml of cell lysate | — |
| Nanoemulsion blank | 4.81 ± 0.38 mg/ml of cell lysate | −1.4% |
| DMSO Lovastatin | 4.71 ± 0.34 mg/ml of cell lysate | −3.5% |
| Nanoemulsion | 1.54 ± 0.18 mg/ml of cell lysate | −68.4% |
| Lovastatin | | |

Hep G-2 cells cultured for 72 hrs. Lovastatin added at 0.45 mM

TABLE 5
HMG CoA Reductase Assay for Hep G2 Cells
Exposed to 0.45 mM Lovastatin for 72 hrs.

| | | | | | |
|---|---|---|---|---|---|
| Group# | 1 | 2 | 3 | 5 | 6 |
| | DMSO | NANO | NANO | DMSO | Control |
| | Lovastatin | Lovastatin | Blank | Alone | Untreated |
| | | | | | cells |

Absorbance Units for Assay Run in Triplicate

| | 1 | 2 | 3 | 5 | 6 |
|---|---|---|---|---|---|
| | 1.49 | −4.156 | 2.163 | 4.6 | 6.3 |
| | 1.49 | −4.859 | 2.111 | 4.7 | 6.3 |
| | 1.29 | −4.499 | 2.111 | 4.859 | 5.4 |
| Mean | 1.423333 | −4.50467 | 2.12833 | 4.719667 | 5.993333 |
| STD | 0.094281 | 0.287027 | 0.02451 | 0.106647 | 0.433692 |
| SEM | 0.054433 | 0.165715 | 0.01415 | 0.061573 | 0.250392 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of forming a nanoemulsion comprising an active component, the method comprising
a. combining a lipophilic component, an active component, a hydrophilic component, and a surfactant in a first composition, wherein the first composition is characterized by a temperature dependent phase inversion between the lipophilic component and the hydrophilic component at or above a phase inversion temperature of the surfactant, and wherein
(i) the first composition contains less than about 5% by weight of the lipophilic component,
(ii) a weight ratio (Ros) of the lipophilic component to the total weight of the surfactant in the first composition is about 0.5 or less,
(iii) the first composition has a weight ratio between the surfactant and the lipophilic component selected to form a nanoemulsion having an average lipophilic component droplet size of up to 100 nm; and
(iv) the active component, the surfactant and the lipophilic component are selected to form a nanoemulsion;
b. heating the first composition above the phase inversion temperature of the surfactant for a time sufficient to cause at least a portion of the first composition to undergo a phase inversion to form a second composition; and
c. allowing the second composition to form a nanoemulsion having droplets of the lipophilic component.

2.-4. (canceled)

5. The method of claim 1, wherein the lipophilic component comprises an oil having at least about 4% of nonsaponifiable components, the surfactant is a polyoxyethylene ester of hydroxysteric acid and the active component is a taxane or benzopyrone.

6. The method of claim 1, wherein the surfactant is a C20 ethoxylated monoglyceride and allowing the second composition to form a nanoemulsion comprises cooling the second composition at a rate effective to form the nanoemulsion without micro fluidization of the second composition.

7. The method of claim 1, wherein the surfactant is selected from the group consisting of: an ethoxylated mono- or diglyceride, a polyoxyethylene ester of hydroxystric acids, a polyoxyethylene sorbitan monooleic acid ester, a polysorbate, a phospholipid, and a polyoxyethylene oil.

8. The method of claim 1, wherein the lipophilic component is an oil selected from the group consisting of: soybean oil, coconut oil, vegetable oil, rice bran oil, and fish oil.

9. The method of claim 1, further comprising the step of dissolving an active component in the lipophilic component prior to forming the first composition.

10. The method of claim 1, wherein the active component comprises one or more of a benzopyrone or a benzopyrone derivative, a polyphenol, a pyrimidine or a pyrimidine analog, an imidazole or an imidazole analog, a taxane, a tocopherol, a tocotrienol, a carotenoid, a polynucleotide, a polypeptide, lutein, and insulin.

11. The method of claim 1, wherein the active component comprises one or more of coumarin, curcumin, 5-fluorouracil, dacarbazine, paclitaxel, vitamin E, lutein, a statin, and insulin.

12. The method of claim 1, wherein the first composition is selected from the group consisting of:
a. an active component comprising coumarin, a polyoxyethylene ester of 12-hydroxysteric acid surfactant and a lipophilic component comprising an oil selected from the group consisting of: soybean oil, fish oil, rice bran oil, and coconut oil;
b. an active component comprising curcumin, a polyoxy ethylene castor oil surfactant and a lipophilic component comprising an oil selected from the group consisting of: soybean oil, fish oil, rice bran oil, and coconut oil;
c. an active component comprising 5-fluorouracil, a polyoxyethylene ester of 12-hydroxysteric acid surfactant and a lipophilic component comprising an oil selected from the group consisting of: soybean oil, fish oil, rice bran oil, and coconut oil;
d. an active component comprising dacarbazine, a polyoxyethylene ester of 12-hydroxysteric acid surfactant and a lipophilic component comprising an oil selected from the group consisting of: soybean oil, fish oil, and coconut oil;
e. an active component comprising paclitaxel, a polyoxyethylene ester of 12-hydroxysteric acid surfactant and a lipophilic component comprising an oil selected from the group consisting of: soybean oil, fish oil, rice bran oil, and coconut oil;
f. an active component comprising a tocotrienol, a polyoxyethylene ester of 12-hydroxysteric acid surfactant and a lipophilic component comprising an oil selected from the group consisting of: soybean oil, fish oil, rice bran oil, and coconut oil;
g. an active component comprising lutein, a C20 ethoxylated monoglyceride surfactant and a lipophilic component comprising an oil selected from the group consisting of: soybean oil, fish oil, and coconut oil; and
h. an active component comprising insulin, a surfactant comprising a combination of Polysorbate 80 and Sorbitan monooleate and a lipophilic component comprising an oil selected from the group consisting of: soybean oil, rice bran oil, fish oil, and coconut oil.

13. A nanoemulsion composition comprising droplets of a lipophilic component suspended in a hydrophilic component and a surfactant present in a total weight ratio of 3:1 to 10:1 with the lipophilic component, wherein
the lipophilic component is less than about 5% of the total weight of the composition,
the nanoemulsion composition has a weight ratio (Ros) of the lipophilic component to the total weight of the surfactant of about 0.5 or less, and
the droplets of the lipophilic component are characterized by a single peak particle size distribution with an average size of up to about 25 nm, and wherein the composition is further characterized by one of the following:
a. the lipophilic component comprises rice bran oil, and the surfactant is a nonionic surfactant comprising a polyoxy ethylene ester of hydroxysteric acid when the active component is a taxane or benzopyrone;
b. the lipophilic component comprises soybean oil and the surfactant is a nonionic surfactant comprising a C20 ethoxylated monoglyceride when the active component comprises a carotenoid; or
c. the lipophilic component comprises an omega-3 oil and the surfactant is a nonionic surfactant comprising a polyoxyethylene ester of hydroxysteric acid when the active component is selected from the group consisting of: a benzopyrone, a pyrimidine, and an imidazole.

14. The composition of claim 13, wherein the surfactant is selected from the group consisting of: an ethoxylated mono- or diglyceride, a polyoxyethylene ester of hydroxystric acids, a polyoxyethylene sorbitan monooleic acid ester, a polysorbate, a phospholipid, and a polyoxyethylene oil.

15. The composition of claim 13, wherein the lipophilic component is an oil selected from the group consisting of: soybean oil, coconut oil, vegetable oil, rice bran oil, and fish oil.

16. (canceled)

17. The composition of claim 13, wherein the surfactant is a nonionic polyethoxylated surfactant and the nanoemulsion further comprises an active component in the lipophilic component.

18. The composition of claim 13, wherein the composition is characterized by at least one of the following:
a. an active component comprising coumarin, and a polyoxyethylene ester of 12-hydroxysteric acid surfactant;
b. an active component comprising curcumin and a polyoxyethylene castor oil surfactant;
c. an active component comprising 5-fluorouracil and a polyoxyethylene ester of 12-hydroxysteric acid surfactant;
d. an active component comprising dacarbazine and a polyoxy ethylene ester of 12-hydroxysteric acid surfactant;
e. an active component comprising paclitaxel and a polyoxyethylene ester of 12-hydroxysteric acid surfactant;
f. an active component comprising a tocotrienol and a polyoxyethylene ester of 12-hydroxysteric acid surfactant;
g. an active component comprising lutein and a C20 ethoxylated monoglyceride surfactant; or
h. an active component comprising insulin, and a surfactant comprising a combination of Polysorbate 80 and Sorbitan monooleate.

19. A method of formulating a medicament comprising a nanoemulsion composition, the method comprising
a. combining a lipophilic component, an active component, a hydrophilic component, and a surfactant in a first composition, wherein the first composition is characterized by a temperature dependent phase inversion between the lipophilic component and the hydrophilic component at or above a phase inversion temperature of the surfactant, and wherein
(i) the first composition contains less than about 5% by weight of the lipophilic component,
(ii) a weight ratio (Ros) of the lipophilic component to the total weight of the surfactant in the first composition is about 0.5 or less, and
(iii) the first composition has a weight ratio between the surfactant and the lipophilic component selected to form a nanoemulsion having an average lipophilic component droplet size of up to 100 nm; and
(iv) the active component, the surfactant and the lipophilic component are selected to form a nanoemulsion;
b. heating the second composition above the phase inversion temperature for a time sufficient to cause at least a portion of the mixture to undergo a phase inversion to form a third composition; and
c. cooling the third composition at a rate effective to form the nanoemulsion having droplets of the lipophilic component of an average droplet size of up to 100 nm suspended in the hydrophilic component; and d. formulating the nanoemulsion as a medicament.

20. The method of claim 19, wherein
a. the surfactant is selected from the group consisting of: an ethoxylated mono- or diglyceride, a polyoxyethylene ester of hydroxystric acids, a polyoxyethylene sorbitan monooleic acid ester, a polysorbate, a phospholipid, and a polyoxyethylene oil;
b. the lipophilic component is an oil selected from the group consisting of: soybean oil, coconut oil, vegetable oil, rice bran oil, and fish oil; and
c. the active component is one or more materials selected from the group consisting of: a benzopyrone or a benzopyrone derivative, a polyphenol, a pyrimidine or a pyrimidine analog, an imidazole or an imidazole analog, a taxane, a tocopherol, a tocotrienol, a carotenoid, a polynucleotide, a polypeptide, lutein, and insulin.

21. The method of claim 19, further comprising:
a. dissolving a lipophilic active component in a non-toxic oil lipophilic component to form an active component composition, the active component being a pharmaceutical, nutraceutical, or a cosmaceutical;
b. combining the active component composition with the hydrophilic component and the surfactant to form the first composition, wherein the active component is more soluble in the lipophilic component than the hydrophilic component;
c. cooling the second composition at a rate effective to form the nanoemulsion having droplets of the lipophilic component of an average droplet size of up to 100 nm suspended in the hydrophilic component, without microfluidizing the second composition;
d. formulating the nanoemulsion formed from the second composition as a medicament; and
e. packaging a therapeutically effective amount of the medicament for administration to a subject in need thereof.

22. A method of forming a nanoemulsion, the method comprising
a. combining a lipophilic component, a hydrophilic component, and a surfactant in a first composition, wherein the first composition is characterized by a temperature dependent phase inversion between the lipophilic component and the hydrophilic component at or above a phase inversion temperature of the surfactant, and wherein
(i) the first composition contains less than about 5% by weight of the lipophilic component,
(ii) a weight ratio (Ros) of the lipophilic component to the total weight of the surfactant in the first composition is about 0.5 or less, and
(iii) the first composition has a weight ratio between the surfactant and the lipophilic component selected to provide a nanoemulsion having an average lipophilic component droplet size of up to 100 nm without micro fluidization;
b. heating the first composition above the phase inversion temperature of the surfactant for a time sufficient to cause at least a portion of the first composition to undergo a phase inversion to form a second composition; and
c. cooling at a rate sufficient for the second composition to form the nanoemulsion having droplets of the lipophilic component without micro fluidizing the second composition.

23. The method of claim 22, wherein the ratio of the lipophilic component to the surfactant is 5:1 or greater and the nanoemulsion has a single distribution of droplet sizes of the lipophilic component having an average droplet size of up to 25 nm and wherein the first composition is selected from the group consisting of:
a. the lipophilic component comprises at least one of rice bran oil and coconut oil and a surfactant selected from the group consisting of: a C20 ethoxylated monoglyceride, a polyoxy ethylene ester of 12-hydroxysteric acid, a polyoxy ethylene sorbitan monooleate, and a polyoxyethylene castor oil;
b. the lipophilic component comprises soybean oil and a surfactant selected from the group consisting of: a C20 ethoxylated monoglyceride, a polyoxyethylene ester of 12-hydroxysteric acid and a polyoxyethylene sorbitan monooleate; and
c. the lipophilic component comprises fish oil and a surfactant selected from the group consisting of: a C20 ethoxylated monoglyceride and a polyoxyethylene ester of 12-hydroxysteric acid.

24. The method of claim 23, wherein the hydrophilic component is water and the composition is characterized by at least one of the following:
a. a ratio of the lipophilic component to the surfactant is 5:1-7:1;
b. a water/(water+oil) weight ratio of up to about 0.980;
c. an oil/(water+oil) weight ratio of up to about 0.023; or
d. a water/(water+surfactant) weight ratio of up to about 0.143.

25. A nanoemulsion composition comprising droplets of a lipophilic component suspended in a hydrophilic component and a nonionic polyethoxylated surfactant present in a total weight ratio of 3:1 to 10:1 with the lipophilic component, wherein
the lipophilic component is less than about 5% of the total weight of the composition,
the nanoemulsion composition has a weight ratio (Ros) of the lipophilic component to the total weight of the surfactant of about 0.5 or less, and
the droplets of the lipophilic component are characterized by a single peak particle size distribution with an average size of up to about 25 nm.

26. A method of decreasing the droplet or particle size of a lipophilic component in a nanoemulsion comprising a hydrophilic component and a surfactant, the method comprising:
a. decreasing the weight ratio (Ros) of the lipophilic component to the total weight of the surfactant and the lipophilic component of the lipophilic component in a first composition further including a nonionic polyethoxylated surfactant and having a phase inversion temperature;
b. heating the first composition above the phase inversion temperature of the first composition for a time sufficient to cause at least a portion of the first composition to undergo a phase inversion to form a second composition; and
c. cooling the second composition at a rate effective to form the nanoemulsion without micro fluidization, the nanoemulsion having droplets of the lipophilic component of an average droplet size of up to 100 nm suspended in the hydrophilic component.

27. (canceled)
